(19) United States
(12) Patent Application Publication
(10) Pub. No.: US 2024/0376176 A1
(43) Pub. Date: Nov. 14, 2024

(54) SYSTEMS AND METHODS FOR DELIVERY BASED ON SUPRAMOLECULAR NANOSTRUCTURES

(71) Applicant: Children's Medical Center Corporation, Boston, MA (US)

(72) Inventors: Tianjiao Ji, Boston, MA (US); Daniel S. Kohane, Newton, MA (US)

(73) Assignee: Children's Medical Center Corporation, Boston, MA (US)

(21) Appl. No.: 18/577,206

(22) PCT Filed: Jul. 8, 2022
(86) PCT No.: PCT/US2022/073533

§ 371 Date: Jan. 5, 2024

Related U.S. Application Data
(60) Provisional application No. 63/219,790, filed on Jul. 8, 2021.

(51) Int. Cl. C07K 14/705

(52) U.S. Cl. CPC C07K 14/705; C07K 2319/00

(57) ABSTRACT

Compositions and methods for the administration of active agents are generally described. In some embodiments, compositions comprising peptide moieties are described. The peptide moieties may comprise sodium channel peptide sequences, such as TQDYWEN (SEQ ID NO: 1) and/or CGEWIET (SEQ ID NO: 2). According to certain embodiments, the peptide moieties are connected to a hydrophobic domain. The presence of both the peptide moiety and the hydrophobic domain may drive self-assembly (e.g., to form a supramolecular nanostructure). The supramolecular nanostructure may facilitate cooperative interaction between distinct peptide sequences. In some embodiments, the peptide sequences bind to the active agents. Binding between the active agent and the peptide sequences may advantageously facilitate controlled release of the active agent. In the context of the present disclosure, it has been inventively recognized that drug delivery may be improved through the use of compositions and methods described herein below.

### RELATED APPLICATIONS

This application claims priority under 35 U.S.C. § 119(e) to U.S. Provisional Application No. 63/219,790, entitled “Systems and Methods for Delivery Based on Supramolecular Nanostructures,” filed on Jul. 8, 2021, the contents of which are incorporated herein by reference in their entirety.

### FEDERALLY SPONSORED RESEARCH

This invention was made with government support under GM131728 awarded by the National Institutes of Health. The government has certain rights in the invention.

### TECHNICAL FIELD

Systems and methods for releasing agents from supramolecular nanostructures are generally described.

### BACKGROUND

Many protein inhibitors have specific interactions with proteins. Peptide sequences from binding sites can bind the protein inhibitors in some cases. Sodium channel blockers are important anesthetic compounds. Some sodium channel peptide sequences can bind sodium channel blockers.

### SUMMARY

Systems and methods for releasing agents from supramolecular nanostructures are generally described. The subject matter of the present disclosure involves, in some cases, interrelated products, alternative solutions to a particular problem, and/or a plurality of different uses of one or more systems and/or articles.

One aspect is generally directed to a composition. In one set of embodiments, the composition comprises a peptide moiety comprising a channel or receptor peptide sequence and a hydrophobic domain connected to the peptide sequence. In another set of embodiments, the composition comprises a peptide moiety comprising a peptide sequence having a structure TQDYWEN (SEQ ID NO: 1) and a hydrophobic domain connected to the peptide sequence. In yet another set of embodiments, the composition comprises a peptide moiety comprising a peptide sequence having a structure CGEWIET (SEQ ID NO: 2) and a hydrophobic domain connected to the peptide sequence.

According to still another set of embodiments, the composition comprises a supramolecular structure, comprising a first peptide moiety comprising a first sodium channel peptide sequence and a first hydrophobic domain, and a second peptide moiety comprising a second sodium channel peptide sequence and a second hydrophobic domain. In some cases, the first hydrophobic domain is associated with the second hydrophobic domain via a hydrophobic interaction.

Another aspect is generally directed to a method. In accordance with one set of embodiments, the method comprises associating an agent with a supramolecular nanofiber comprising a first peptide moiety and a second peptide moiety, and releasing the agent from the supramolecular nanofiber, e.g., over a period of at least 3 hours.

In another set of embodiments, the method comprises associating an agent with a supramolecular nanostructure comprising a first peptide moiety comprising a first peptide sequence and a first hydrophobic domain, and a second peptide moiety comprising a second peptide sequence and a second hydrophobic domain, and releasing the agent from the supramolecular structure, e.g., over a period of at least 3 hours. In some cases, the first hydrophobic domain is associated with the second hydrophobic domain via a hydrophobic interaction.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIGS. 1A-1C present exemplary peptide moieties, according to some embodiments;

FIGS. 2A-2B present exemplary cross-sectional schematic illustrations of supramolecular nanostructures comprising peptide moieties, in some embodiments;

FIGS. 3A-3E present exemplary perspective schematic illustrations of supramolecular nanostructures, according to certain embodiments;

FIG. 4A presents an exemplary illustration of a site 1 sodium channel, according to certain embodiments;

FIG. 4B presents an exemplary agent, according to certain embodiments;

FIG. 4C presents an exemplary agent, according to certain embodiments;

FIG. 4D presents exemplary peptide sequences, according to certain embodiments;

FIG. 5 presents exemplary data on the release kinetics of TTX, in solution or from peptide moieties, according to certain embodiments;

FIG. 6 presents exemplary data on release kinetics of an exemplary agent, according to certain embodiments;

FIG. 7A presents exemplary transmission electron microscopy (TEM) images of peptide moiety solutions with and without TTX, according to certain embodiments;

FIG. 7B presents exemplary data on the size distribution of peptide moieties in solution, according to certain embodiments;

FIG. 8 presents exemplary structures of peptide moieties with different hydrophobic domains according to certain embodiments;

FIG. 9 presents exemplary data used to compute the critical micelle concentration (CMC) peptide moieties, according to certain embodiments;

FIG. 10 presents exemplary TEM images of supramolecular nanostructures, according to certain embodiments according to certain embodiments;

FIG. 11 presents the measured average width of supramolecular nanofibers based on the TEM images, according to certain embodiments;

FIG. 12 presents an exemplary TEM image of peptide nanostructures in the presence of TTX, according to certain embodiments;

FIG. 13 presents an exemplary comparison of the measured average width of nanofibers in the absence or presence of TTX, according to certain embodiments;

FIG. 14 presents cumulative TTX release from solutions comprising supramolecular nanostructures, according to certain embodiments according to certain embodiments;

FIG. 15 presents exemplary data of the release kinetics of an agent in solution or from peptide moieties, according to certain embodiments;

FIG. 16A presents exemplary TEM images of supramolecular nanofibers, according to certain embodiments;

FIG. 16B presents exemplary TTX release kinetics of an agent from supramolecular nanostructures, according to certain embodiments;

FIG. 17A presents molecular structures and sequences of peptide moieties, according to certain embodiments;

FIG. 17B presents exemplary TEM images of supramolecular nanostructures, according to certain embodiments;

FIG. 17C presents release kinetics of TTX from supramolecular nanostructures, according to certain embodiments;

FIG. 18 presents exemplary circular dichroism spectra of exemplary peptide moieties comprised by exemplary supramolecular nanostructures according to certain embodiments;

FIG. 19 presents exemplary structures of peptide moieties, according to certain embodiments;

FIG. 20A presents exemplary TEM images of exemplary supramolecular nanostructures, according to certain embodiments;

FIG. 20B presents an exemplary graph of tetrodotoxin (TTX) release from exemplary solutions comprising peptide moieties, according to certain embodiments;

FIG. 21 presents exemplary data on the critical micelle concentration (CMC) of exemplary peptide moieties, according to certain embodiments;

FIG. 22 presents an exemplary schematic of a competitive binding assay, according to certain embodiments;

FIG. 23 presents exemplary TEM images of solutions comprising peptide moieties, according to certain embodiments;

FIGS. 24A-24B presents exemplary data on the assessment of whether peptides moieties interfered with an ELISA assay, according to certain embodiments;

FIGS. 25A-25B present exemplary measurements of Kd of peptide nanofibers with TTX, according to certain embodiments;

FIG. 26 presents an exemplary equation for computing Kd, according to certain embodiments;

FIG. 27A presents exemplary data on the release kinetics of an exemplary agent and a varying concentration of an exemplary peptide moiety, according to certain embodiments;

FIG. 27B presents exemplary data from FIG. 27A at 12 h to show relationship between the exemplary peptide moiety concentration and release of the exemplary agent, according to certain embodiments;

FIG. 27C presents TEM micrographs of various concentrations of exemplary peptide moieties, according to certain embodiments;

FIG. 28 presents exemplary data on the cytotoxicity of peptide moiety formulations, according to certain embodiments;

FIG. 29A presents exemplary data on the duration of sensory nerve blocks according to certain embodiments;

FIG. 29B presents exemplary data on the thermal latency in the uninjected (contralateral) extremity in the first 5 h after injection according to certain embodiments;

FIG. 30 presents exemplary micrographs showing tissue reaction to peptide moieties 4 days after injection, according to certain embodiments;

FIG. 31 presents exemplary images of the sciatic nerves and adjacent tissues of rats 14 days after injection with exemplary peptide moieties and agents, according to certain embodiments;

FIG. 32 presents exemplary TEM images of supramolecular nanostructures, according to certain embodiments;

FIG. 33 presents exemplary data on the representative time courses of retention of formulations at the site of injection, detected by an in vivo imaging system, according to certain embodiments;

FIG. 34 presents exemplary data on the quantification of the fluorescence intensity of labeled peptide moieties over time, according to certain embodiments;

FIG. 35 presents exemplary laser scanning confocal microscopy images of sciatic nerve in rats injected with doses of peptide moieties, according to certain embodiments;

FIG. 36 presents exemplary data on the viscosity of solutions comprising peptide moieties, according to certain embodiments;

FIG. 37 presents exemplary data on the comparison of tissue retention half-life and duration of nerve block of formulations, according to certain embodiments;

FIG. 38 presents exemplary data on the release kinetics of the exemplary agent saxitoxin (STX) from formulations with and without peptide moieties, according to certain embodiments;

FIGS. 39A-39B present exemplary in vivo data on the duration and thermal latency of sensory nerve blocks after injection with an exemplary agent, with and without peptide moieties, according to certain embodiments;

FIG. 40 presents exemplary data on the duration of sensory nerve block from the exemplary agent dicarbamoyl saxitoxin (dcSTX), with and without peptide moieties, according to certain embodiments;

FIG. 41 presents exemplary micrographs of sciatic nerves of rats after injection with exemplary agents and/or peptide moieties, according to certain embodiments; and

FIGS. 42A-42B present exemplary data on release and duration of nerve blocks of an exemplary agent, according to certain embodiments.

### DETAILED DESCRIPTION

Compositions and methods for the administration of active agents are generally described. In some embodiments, compositions comprising peptide moieties are described. The peptide moieties may comprise sodium channel peptide sequences, such as TQDYWEN (SEQ ID NO: 1) and/or CGEWIET (SEQ ID NO: 2). According to certain embodiments, the peptide moieties are connected to a hydrophobic domain. The presence of both the peptide moiety and the hydrophobic domain may drive self-assembly (e.g., to form a supramolecular nanostructure). The supramolecular nanostructure may facilitate cooperative interaction between distinct peptide sequences. In some embodiments, the peptide sequences bind to the active agents. Binding between the active agent and the peptide sequences may advantageously facilitate controlled release of the active agent. In the context of the present disclosure, it has been inventively recognized that drug delivery may be improved through the use of compositions and methods described herein below.

Several specific, practical advantages may be best illustrated by a specific, exemplary embodiment. In one example, a supramolecular nanostructure has a first sodium channel peptide sequence and a second sodium channel peptide sequence, and each sodium channel peptide sequence is connected to a hydrophobic domain. In some cases, the first sodium channel peptide sequence and the second sodium channel peptide sequence bind to an agent, such as a sodium channel blocker. The first sodium channel peptide sequence and the second sodium channel peptide sequence can, in some cases, have a higher binding affinity for the agent when used together rather than individually (i.e., the first peptide sequence and the second peptide sequence may associate with an agent cooperatively). The first sodium channel peptide sequence and the second sodium channel peptide sequence can also have a higher binding affinity for the agent, in some cases, when used together but assembled into supramolecular nanostructures comprising one of the peptide sequences in a significantly greater concentration than the other. This cooperative association with the agent may be further enhanced by the incorporation of both peptide sequences into the supramolecular nanostructure. For example, without wishing to be bound by theory, the nanostructure may help to retain the first sodium channel peptide sequence and the second sodium channel peptide sequence in close proximity and/or may increase the likelihood of finding the first and the second sodium peptide channel sequences in an appropriate configuration (e.g., conformation) for binding to the agent.

Thus, the supramolecular nanostructure may be used to release the agent, e.g., to a patient or a subject, as described in greater detail below. The release of an agent from such a nanostructures can be advantageous, requiring less frequent dosing, and in some cases reducing the risk associated with an overdose of the agent. It should, of course, be understood that the forgoing example is a specific embodiment that should not be considered limiting, and that broader or alternative embodiments, applications, and advantages are described elsewhere herein.

According to some aspects, the disclosure is directed towards a composition. In some embodiments, the composition comprises a peptide moiety. The peptide moiety may comprise a peptide sequence (e.g., a sodium channel peptide sequence). In some embodiments, the peptide moiety further comprises a hydrophobic domain, described in greater detail herein below. The hydrophobic domain is connected to the peptide sequence, in some embodiments. In some embodiments, the composition comprises a supramolecular nanostructure. In some embodiments, a method comprises associating an agent with the supramolecular nanostructure. In some embodiments, a method comprises releasing the agent from the supramolecular nanostructure.

In some embodiments, the peptide moiety comprises a peptide sequence. The peptide sequence may be any suitable peptide sequence. In some embodiments, it may be advantageous for the peptide sequence to be hydrophilic, e.g., so that it may act as a hydrophilic domain as described in greater detail below. Hydrophilicity of the peptide sequence may, advantageously, facilitate self-assembly of the peptide moiety into a supramolecular nanostructure.

According to some embodiments, the peptide sequence comprises a channel peptide. For example, in some embodiments, the peptide sequence comprises a sodium channel peptide. In some embodiments, the peptide sequence comprises a receptor peptide. The sequence may have a structure TQDYWEN (SEQ ID NO: 1). As another example, in some embodiments, the sequence has a structure CGEWIET (SEQ ID NO: 2). As yet another example, in some embodiments, the sequence has a structure TFKGWTI (SEQ ID NO: 25). As another example, in some embodiments the sequence has a structure TSAGWDG (SEQ ID NO: 26). In some embodiments, a composition comprises multiple peptide sequences. For example, composition may comprise a peptide moiety comprising multiple peptide sequences (e.g., SEQ ID NO: 1, SEQ ID NO: 2). In some embodiments, the composition comprises more than one (e.g., at least 2, at least 3, at least 4, at least 5, at least 6, or more) peptide moieties. In some embodiments, the more than one peptide moieties comprises a first peptide moiety comprising a first peptide sequence and a second peptide moiety comprising a second peptide sequence. For example, the first peptide moiety may have a sequence having a structure TQDYWEN (SEQ ID NO: 1), and the second peptide moiety may have a sequence having a structure CGEWIET (SEQ ID NO: 2).

In some embodiments, a peptide sequence can be associated with (e.g., bind to) an agent. For example, in some embodiments two or more peptide sequences can be associated with an agent (e.g., simultaneously). According to certain embodiments, the presence of a first peptide sequence and a second peptide sequence in a composition (e.g., as part of the same peptide moiety, or as part of different peptide moieties) advantageously allows the first peptide sequence and the second peptide sequence to simultaneously associate with an agent. In some embodiments, the presence of both the first peptide sequence and the second peptide sequence increases retention of the agent by the composition, relative to the retention that would be observed in the presence of only the first peptide sequence or the second peptide sequence.

In some embodiments, a peptide moiety comprises a hydrophobic domain. Examples of hydrophobic domains include long aliphatic chains (e.g., saturated, unsaturated aliphatic chains), hydrophobic peptide sequences, polycyclic compounds (e.g., compounds comprising 3 or more closed rings of carbon), certain aromatic compounds, steroids, and hydrophobic polymers (e.g., polystyrene). As another example, the hydrophobic domain may be connected to a peptide sequence of the peptide moiety. Typically, the hydrophobic domain is not inherently part of a naturally occurring amino acid (for example, not a side chain of a naturally-occurring amino acid), but is instead connected to the peptide sequence, e.g., via a covalent bond.

In some embodiments, the hydrophobic domain is connected to the peptide covalently. For example, in some embodiments, the hydrophobic domain is a C-terminal modification of the peptide sequence. As another example, in some embodiments, the hydrophobic domain is an N-terminal modification of the peptide sequence. As yet another example, in some embodiments, the hydrophobic domain is a sidechain modification of the residue of the peptide sequence. However, the hydrophobic domain may be noncovalently connected to the peptide sequence. For example, the hydrophobic domain may be connected to the peptide sequence via intermolecular interactions such as hydrogen bonds. In some embodiments, the hydrophobic peptide sequence is connected to a peptide sequence at a first terminus of the hydrophobic peptide sequence. The hydrophobic domain may be connected to a second peptide sequence (e.g., at a second terminus of the hydrophobic peptide sequence). For example, in some embodiments, the hydrophobic domain is connected to a second peptide sequence comprising a sequence having the structure of TQDYWEN (SEQ ID NO: 1) or CGEWIET (SEQ ID NO: 2).

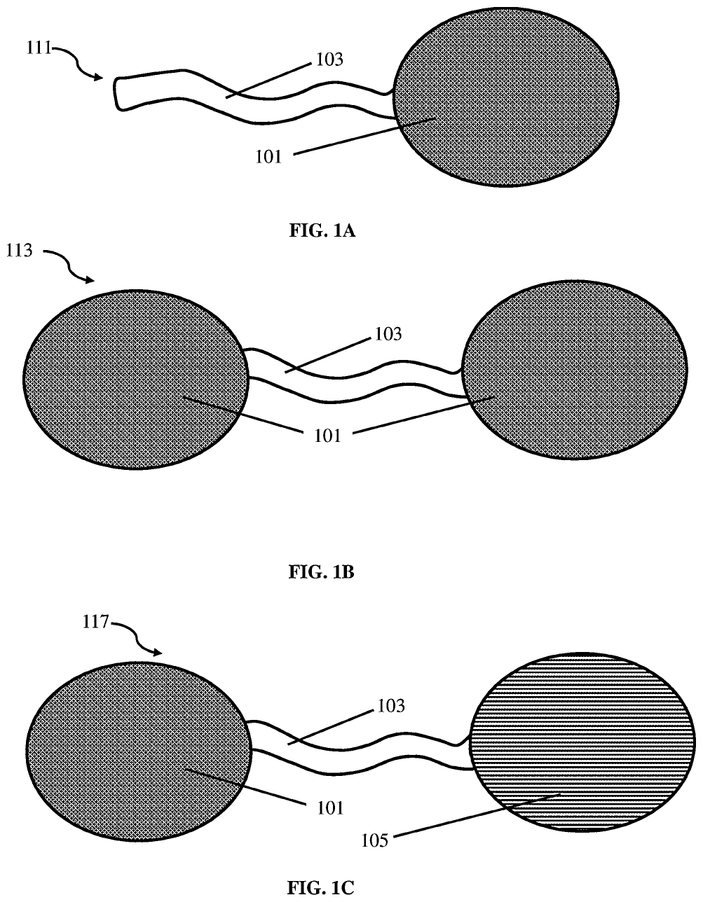

FIGS. 1A-1C present exemplary peptide moieties, according to certain embodiments. For example, FIG. 1A presents exemplary peptide moiety 111, comprising peptide sequence 101 connected to hydrophobic domain 103, in some embodiments. FIG. 1B presents an alternative embodiment of a peptide moiety 113, comprising two identical peptide sequences 101, both connected to hydrophobic domain 103. FIG. 1C presents another embodiment of a peptide moiety 117, comprising first peptide sequence 101 and second peptide sequence 105, both connected to hydrophobic domain 103.

The hydrophobic domain may have any appropriate composition. In some embodiments, the hydrophobic domain comprises a hydrophobic peptide sequence. The hydrophobic peptide sequence may comprise hydrophobic amino acids. Examples of hydrophobic amino acids include: hydrophobic, naturally-occurring amino acids such as alanine, isoleucine, leucine, methionine, phenylalanine, tryptophan, tyrosine, valine, glycine, and proline; and hydrophobic, non-naturally-occurring amino acids. Of course, the hydrophobic peptide sequence is not limited to hydrophobic amino acid residues, and may comprise other amino acid residues. In some embodiments, the hydrophobic peptide sequence comprises between 3 and 10 hydrophobic amino acid residues. For example, the hydrophobic peptide sequence comprises 3, 4, 5, 6, 7, 8, 9, or 10 hydrophobic amino acids, according to some embodiments. In some embodiments, greater than or equal to 50%, greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, and/or up to 100% of an amino acid residues within the hydrophobic amino acid sequence are hydrophobic amino acids. However, embodiments wherein less than 50% of the amino acid residues in the hydrophobic amino acid sequence are also contemplated. Of course, embodiments of hydrophobic peptide sequences comprising fewer than 3 or greater than 10 hydrophobic amino acids are also contemplated, and the disclosure is not thus limited. In some embodiments, the hydrophobic amino acids are consecutive in the hydrophobic amino acid sequence. However, the hydrophobic amino acids may be separated by one or more non-hydrophobic amino acid residues. In some embodiments, the hydrophobic peptide sequence is connected to a second peptide sequence comprising a sequence having the structure of TQDYWEN (SEQ ID NO: 1) or CGEWIET (SEQ ID NO: 2).

Generally, it should be understood that while a hydrophobic domain may comprise a hydrophobic amino acid (e.g., as part of a hydrophobic amino acid sequence), a single, naturally-occurring hydrophobic amino acid is not considered to be or comprise a hydrophobic domain. Generally, a hydrophobic domain may comprise more than a single, naturally-occurring amino acid.

In some embodiments, the hydrophobic domain comprises a species of type (I)

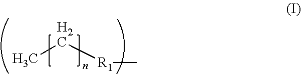

wherein R₁ is a carbonyl carbon or R₁ is an amide nitrogen, and wherein n is greater than or equal to 1 and less than or equal to 30 (e.g., wherein n=1, 2, 3, 4, 5, 6, 7, 8, 9, 10, 11, 12, 13, 14, 15, 16, 17, 18, 19, 20, 21, 22, 23, 24, 25, 26, 27, 28, 29, or 30). For example, in some embodiments, the species of type (I) is connected to the peptide sequence via a C-terminal amidation of the peptide sequence and R₁ is an amide nitrogen. As another example, in some embodiments, the species of type (I) is connected to the peptide sequence via an N-terminal amidation of the peptide sequence and R₁ is a carbonyl carbon. In some embodiments, the species of type (I) is connected to the peptide sequence via amidation of a side-chain of the peptide sequence, such as a lysine side-chain, an aspartic acid side-chain, or a glutamic acid side-chain of the peptide sequence. The species of type (I) may also be connected to the peptide sequence via esterification of a side-chain of the peptide sequence, such as a serine side-chain or an threonine side-chain of the peptide sequence. Embodiments where n is greater than 30 are also contemplated.

In some embodiments, the hydrophobic domain comprises a species of type (II)

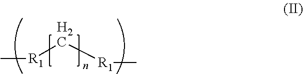

where R₁ and R₂ are each, independently selected from a carbonyl carbon and an amide nitrogen, wherein R₂ is bonded to a second peptide sequence, and wherein n is greater than or equal to 1 and less than or equal to 30 (e.g., wherein n=1, 2, 3, 4, 5, 6, 7, 8, 9, 10, 11, 12, 13, 14, 15, 16, 17, 18, 19, 20, 21, 22, 23, 24, 25, 26, 27, 28, 29, or 30). For example, in some embodiments, the hydrophobic domain comprises a species of type (II) where R₁ and R₂ are each, independently selected from a carbonyl carbon and an amide nitrogen, and wherein R₂ is bonded to a second peptide sequence comprising a sequence having the structure of TQDYWEN (SEQ ID NO: 1) or CGEWIET (SEQ ID NO: 2). Embodiments where n is greater than 30 are also contemplated.

In some embodiments, the species of type (II) is connected to the first peptide sequence via a C-terminal amidation of the first peptide sequence and R₁ is an amide nitrogen. As another example, in some embodiments, the species of type (II) is connected to the first peptide sequence via an N-terminal amidation of the first peptide sequence and R₁ is a carbonyl carbon. In some embodiments, the species of type (II) is connected to the first peptide sequence via amidation of a side-chain of the first peptide sequence, such as a lysine side-chain, an aspartic acid side-chain, or a glutamic acid side-chain of the first peptide sequence.

In some embodiments, the species of type (II) is connected to the second peptide sequence via a C-terminal amidation of the second peptide sequence and R₂ is an amide nitrogen. As another example, in some embodiments, the species of type (II) is connected to the second peptide sequence via an N-terminal amidation of the second peptide sequence and R₂ is a carbonyl carbon. In some embodiments, the species of type (II) is connected to the second peptide sequence via amidation of a side-chain of the first peptide sequence, such as a lysine side-chain, an aspartic acid side-chain, or a glutamic acid side-chain of the second peptide sequence.

In some embodiments, a peptide moiety comprises a hydrophilic domain. The hydrophilic domain may, for example, comprise a peptide sequence such as a sodium channel peptide, as described in greater detail elsewhere herein. In some embodiments, the hydrophilic domain comprises hydrophilic amino acid residues. Examples of hydrophilic amino acids include: hydrophilic, naturally occurring amino acids such as arginine, histidine, lysine, aspartic acid, glutamic acid, serine, threonine, spa arginine, glutamine, and glycine; and hydrophilic, non-naturally occurring amino acids. Of course, the peptide sequence is not limited to hydrophilic amino acid residues, and may comprise other amino acid residues. In some embodiments, the hydrophilic domain comprises between 1 and 10 hydrophilic amino acid residues. For example, the hydrophilic domain comprises 1, 2, 3, 4, 5, 6, 7, 8, 9, or 10 hydrophilic amino acids, according to some embodiments. In some embodiments, the hydrophobic amino acids are consecutive in the hydrophilic domain. However, the hydrophilic amino acids may be separated by one or more non-hydrophilic amino acid residues, in some embodiments. Of course, embodiments of hydrophilic domains comprising more than 10 hydrophobic amino acids are also contemplated, and the disclosure is not thus limited.

In one aspect, the composition comprises a supramolecular nanostructure. For example, in some embodiments, a peptide moiety as described elsewhere herein is assembled into the supramolecular nanostructure. The supramolecular nanostructure, according to some embodiments, comprises a peptide moiety (e.g., a first peptide moiety, a second peptide moiety). In some embodiments, the peptide moiety comprises a peptide sequence (e.g., a first peptide sequence, a second peptide sequence) as described elsewhere herein in greater detail. The peptide sequence may be, for example, part of a hydrophilic domain of a peptide moiety. According to certain embodiments, the peptide moiety further comprises a hydrophobic domain (e.g., a first hydrophobic domain, a second hydrophobic domain) as described elsewhere herein in greater detail.

In some embodiments, the supramolecular nanostructures comprises both a first peptide moiety and a second peptide moiety. In some embodiments, the supramolecular nanostructure comprises the first peptide moiety in a proportion of greater than or equal to 1 molar percent (mol %), greater than or equal to 5 mol %, greater than or equal to 10 mol %, greater than or equal to 25 mol %, greater than or equal to 50 mol %, greater than or equal to 75 mol %, or greater. In some embodiments, the supramolecular nanostructure comprises the first peptide moiety in a proportion of greater than or equal to 99 mol %, less than or equal to 95 mol %, less than or equal to 90 mol %, less than or equal to 75 mol %, less than or equal to 50 mol %, less than or equal to 25 mol %, or less. Combinations of these ranges are possible. For example, in some embodiments, the supramolecular nanostructure comprises the first peptide moiety in a proportion of greater than or equal to 1 mol % and less than or equal to 99 mol %. As a more specific embodiment, the supramolecular nanostructure comprises the first peptide moiety in a proportion of greater than or equal to 25 mol % and less than or equal to 75 mol %.

In some embodiments, the supramolecular nanostructure comprises the second peptide moiety in a proportion of greater than or equal to 1 mol %, greater than or equal to 5 mol %, greater than or equal to 10 mol %, greater than or equal to 25 mol %, greater than or equal to 50 mol %, greater than or equal to 75 mol %, or greater. In some embodiments, the supramolecular nanostructure comprises the second peptide moiety in a proportion of greater than or equal to 99 mol %, less than or equal to 95 mol %, less than or equal to 90 mol %, less than or equal to 75 mol %, less than or equal to 50 mol %, less than or equal to 25 mol %, or less. Combinations of these ranges are possible. For example, in some embodiments, the supramolecular nanostructure comprises the second peptide moiety in a proportion of greater than or equal to 1 mol % and less than or equal to 99 mol %.

In some embodiments, the supramolecular nanostructure comprises one or more amphiphiles. For example, the first peptide moiety may be an amphiphile, and/or the second peptide moiety may be an amphiphile. In some embodiments, the supramolecular nanostructure comprises 1, 2, 3, 4, 5 or more species of peptide moieties, according to some embodiments. For example, the supramolecular nanostructure may comprise a first peptide moiety, a second peptide moiety, a third peptide moiety, a fourth peptide moiety, and/or a fifth peptide moiety. The first peptide moiety, the second peptide moiety, the third peptide moiety, the fourth peptide moiety, and/or the fifth peptide moiety may be amphiphiles. In some embodiments, the supramolecular nanostructure comprises amphiphiles that are not peptide moieties.

An amphiphile, in some embodiments, comprises a hydrophobic domain and a hydrophilic domain, as described in greater detail elsewhere herein. In certain embodiments, when added to an aqueous solution, the amphiphile will spontaneously self-assemble to form a supramolecular nanostructure. Examples of supramolecular nanostructures may include a micelle, a vesicle, a nanofiber, a monolayer, a hollow tube, or a ribbon (e.g., a flat ribbon, a helical ribbon). It should be understood that, while these supramolecular nanostructures are common, other types of nanostructures can also form, and that the disclosure is not so limited. The supramolecular nanostructure may be determined by any one of a variety of methods familiar to those of ordinary skill in the art, such as transmission electron microscopy (e.g., negative stain transmission electron microscopy, cryogenic transmission electron microscopy), scanning electron microscopy (SEM), atomic force microscopy (AFM), or X-ray scattering (e.g., small angle X-ray scattering, wide angle X-ray scattering).

In some embodiments, the supramolecular nanostructure comprises a hydrophilic region, adjacent to the aqueous solution, and a hydrophobic region, separated from the aqueous solution by the hydrophilic region. According to certain embodiments, the hydrophobic domain of a self-assembled amphiphile will tend to remain within the hydrophobic region of the supramolecular nanostructure (e.g., within the core of the nanostructure). In some embodiments, hydrophobic domains of amphiphiles are associated by hydrophobic interactions. For example, in some embodiments, a first hydrophobic domain of a first peptide moiety is associated with a second hydrophobic domain of a second peptide moiety via a hydrophobic interaction.

According to certain embodiments, the hydrophilic domain of a self-assembled amphiphile will tend to remain within the hydrophilic region of the self-assembled supramolecular nanostructure. This may allow at least a part of the hydrophilic domain to remain in contact with the aqueous solution, while reducing contact between the aqueous solution and the hydrophobic domain. In some embodiments, hydrophilic domains are associated by hydrophilic interactions. For example, in some embodiments, a first hydrophilic domain of a first peptide moiety is associated with a second hydrophilic domain of a second peptide moiety via a hydrophilic interaction.

In some embodiments, a peptide moiety may comprise at least two hydrophilic domains, connected by a hydrophobic domain. According to certain embodiments, a peptide moiety comprising at least two hydrophilic domains, connected by a hydrophobic domain, is a bolaamphiphile. Referring again to FIGS. 1B-1C, peptide moiety 113 in FIG. 1B and peptide moiety 117 in FIG. 1C can be considered bolaamphiphiles, in some embodiments. For example, in some embodiments a bolaamphiphile comprises a hydrophobic domain that comprises a species of type (II), described above, connected to a first hydrophilic domain via a covalent attachment of the first hydrophilic domain to R₁, and connected to a second hydrophilic domain via a covalent attachment of the second hydrophilic domain to R₂.

In some embodiments, a supramolecular nanostructure may comprise bolaamphiphiles. Bolaamphiphiles, in some embodiments, modify and/or mechanically support a supramolecular self-assembly. In some embodiments, a supramolecular assembly may comprise only bolaamphiphiles. In some embodiments, a first peptide moiety (e.g., comprising a first sodium channel peptide sequence) and a second peptide moiety (e.g., comprising a second sodium channel peptide sequence) of the supramolecular assembly are both bolaamphiphiles. In some embodiments, the first peptide moiety and the second peptide moiety may be different. In some embodiments, the first peptide moiety and the second peptide moiety may be the same (e.g., the first peptide moiety and the second peptide moiety may each comprise a hydrophilic domain, a first sodium channel peptide sequence, and a second sodium channel peptide sequence).

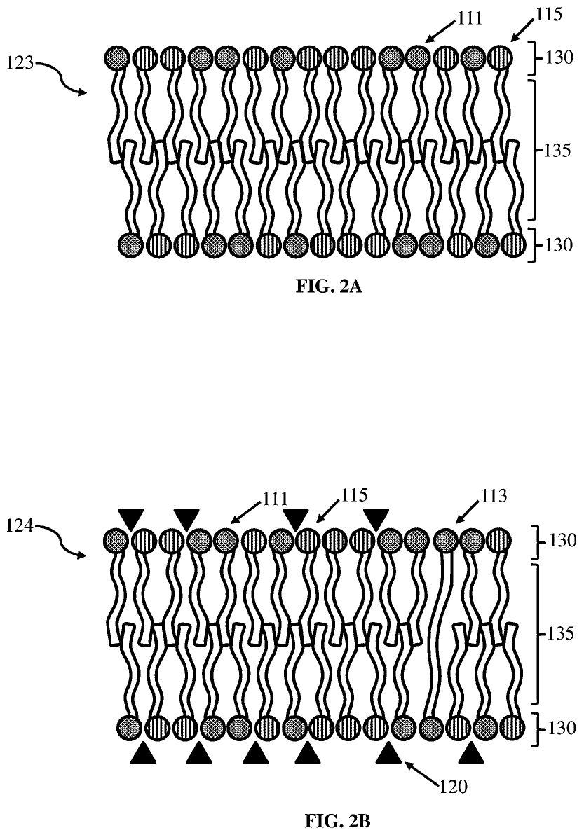

These principles are exemplified by FIGS. 2A-2B, which present cross-sectional schematic illustrations of supramolecular nanostructures, according to some embodiments. In FIG. 2A, supramolecular nanostructure 123 comprises first peptide moiety 111 and second peptide moiety 115. In some embodiments, such as that of FIG. 2A, the supramolecular nanostructure comprises at least one hydrophilic region 130. In some embodiments, such as that of FIG. 2A, the supramolecular nanostructure comprises a hydrophobic domain 135. FIG. 2B is similar to FIG. 2A, except that in the example of FIG. 2B, supramolecular nanostructure 124 comprises peptide moiety 113 (which is a bolaamphiphile, in some embodiments) and is associated with agent 120, as described in greater detail below. While peptide moiety 113 in FIG. 2B is shown traversing the hydrophilic domain, it should be understood that this is non-limiting, and that in some embodiments, similar peptide moieties may bend (e.g., into a U-shape), such that two or more hydrophilic domains of the peptide moiety are positioned within the same hydrophilic region 130, rather than in separate hydrophilic regions.

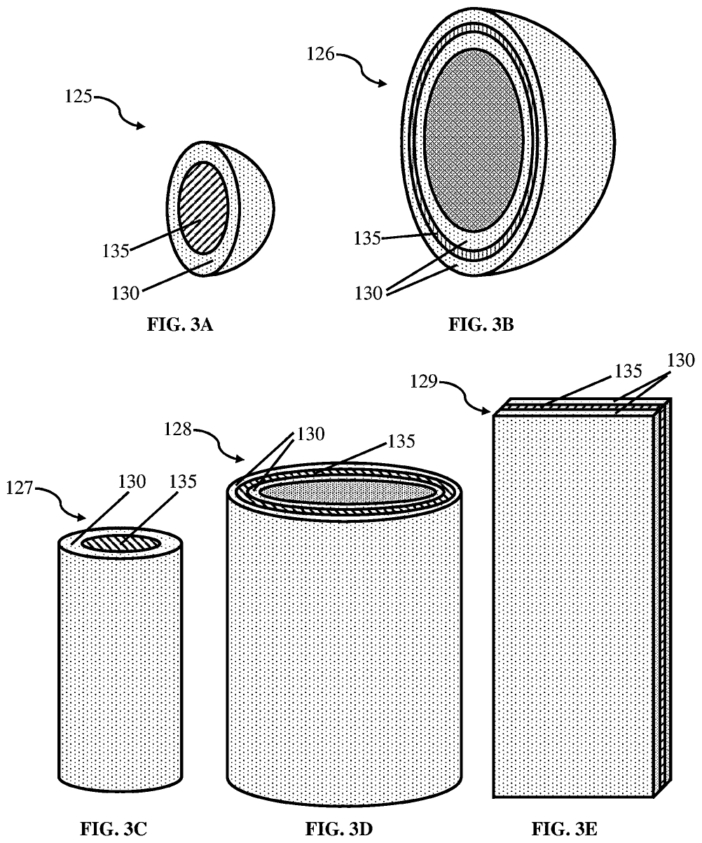

FIGS. 3A-3E present exemplary illustrations of supramolecular nanostructures, according to certain embodiments. In each of FIGS. 3A-3E, the supramolecular nanostructure comprises hydrophobic region 135 and at least one hydrophilic region 130. In some embodiments, the hydrophobic domain of an amphiphile will tend to segregate to hydrophobic region 135, while the hydrophilic domain of an amphiphile will tend to segregate to one of hydrophilic regions 130. As in FIG. 3A, in some embodiments the supramolecular nanostructure is a micelle 125 (note that for visual clarity, FIG. 3A shows half of a spherical micelle, rather than a complete micelle). As in FIG. 3B, in some embodiments the supramolecular nanostructure is a vesicle 126 (note that for visual clarity, FIG. 3B shows half of a spherical vesicle, rather than a complete vesicle). As in FIG. 3C, in some embodiments the supramolecular nanostructure is a nanofiber 127. As in FIG. 3D, in some embodiments the supramolecular nanostructure is a hollow tube 128. As in FIG. 3E, in some embodiments the supramolecular nanostructure is a ribbon 129.

In some embodiments, supramolecular nanostructures comprising peptide moieties may acquire structure at least partially as a result of intramolecular interactions between peptide sequences of the peptide moieties. A supramolecular nanostructure may comprise peptides having elements of protein secondary structure. For example, a supramolecular nanostructure may have beta-sheet character, in some embodiments. In some embodiments, a supramolecular nanostructure may have alpha sheet character. In some embodiments, the supramolecular nanostructure does not comprise peptides having elements of protein secondary structure. For example, in some embodiments, peptides within a supramolecular nanostructure principally have a random coil structure. In some cases, the random coil structure advantageously improves the ability of the first peptide moiety and the second peptide moiety to bind to the agent. Without wishing to be bound by theory, it is believed that the random coil structure may more easily facilitate cooperative interactions between the first and the second peptide moieties by allowing greater freedom of motion of the peptide moieties.

A secondary structure of a supramolecular nanostructure may be characterized using circular dichroism measurements. Circular dichroism may be performed at any suitable concentration. In some embodiments, circular dichroism may be performed at a concentration of greater than or equal to 5 micromolar, greater than or equal to 10 micromolar, greater than or equal to 20 micromolar, greater than or equal to 30 micromolar, greater than or equal to 50 micromolar, greater than or equal to 100 micromolar, or greater. In some embodiments, circular dichroism may be performed at a concentration of less than or equal to 500 micromolar, less than or equal to 200 micromolar, less than or equal to 100 micromolar, less than or equal to 50 micromolar, less than or equal to 30 micromolar, less than or equal to 20 micromolar, or less. Combinations of these ranges are possible. For example, in some embodiments, circular dichroism is performed at a concentration of greater than or equal to 5 micromolar and less than or equal to 200 micromolar.

According to certain embodiments, circular dichroism analysis of the supramolecular nanostructure produces a mean residue ellipticity of greater than or equal to −10, greater than or equal to −8, greater than or equal to −6, greater than or equal to −4, greater than or equal to −2, greater than or equal to 0, or greater. In some embodiments, circular dichroism analysis of the supramolecular nanostructure produces a mean residue ellipticity of less than or equal to 10, less than or equal to 8, less than or equal to 6, less than or equal to 4, less than or equal to 2, less than or equal to 0, or less. Combinations of these ranges are possible. For example, in some embodiments, circular dichroism analysis of the supramolecular nanostructure produces a mean residue ellipticity of greater than or equal to −10 and less than or equal to 10. In some embodiments, a mean residue ellipticity falling within these ranges may indicate a random coil character for peptides in the supramolecular nanostructure.

In some embodiments, circular dichroism is performed for wavelengths of greater than or equal to 190 nm, greater than or equal to 195 nm, greater than or equal to 200 nm, greater than or equal to 205 nm, greater than or equal to 210 nm, greater than or equal to 215 nm, or greater. In some embodiments, circular dichroism is performed for wavelengths of less than or equal to 260 nm, less than or equal to 255 nm, less than or equal to 250 nm, less than or equal to 245 nm, less than or equal to 240 nm, less than or equal to 235 nm, or less. Combinations of these ranges are possible. For example, in some embodiments, circular dichroism is performed for wavelengths of greater than or equal to 190 nm and less than or equal to 260 nm.

Combinations of the forgoing ranges are also possible. For example, in some embodiments, circular dichroism analysis of the supramolecular nanostructures at a concentration of greater than or equal to 20 micromolar amphiphile produces a mean residue ellipticity of greater than or equal to −10 and less than or equal to 10 for wavelengths greater than or equal to 190 nm and less than or equal to 260 nm.

As used herein, a supramolecular nanostructure comprises a plurality of amphiphiles (e.g., peptide moieties) that form the supramolecular nanostructure, at least in part, as a result of intermolecular forces (e.g., hydrogen bonding, dipole-dipole interactions, dipole-induced dipole interactions, and/or van der Waals forces) between the plurality of amphiphiles. In some embodiments, the plurality of amphiphiles are not connected to one another by covalent bonds. However, embodiments wherein the amphiphiles are covalently cross-linked to one another are contemplated.

In some embodiments, a peptide moiety is associated with (e.g., bound to) an agent. For example, the agent may be associated with a first peptide sequence (e.g., a first sodium channel peptide sequence) comprised by the first peptide moiety and/or a second peptide sequence (e.g., a second sodium channel peptide sequence) comprised by a second peptide moiety. In some embodiments, the agent may be bound noncovalently. In some embodiments, as illustrated in FIG. 2B, the agent (e.g., agent 120) can associate with both a first peptide moiety (e.g., first peptide moiety 111) and a second peptide moiety (e.g., second peptide moiety 115). In some embodiments, the agent is associated with a supramolecular nanostructure. In some embodiments, the agent may be deliberately associated with (e.g., bound to) the peptide moiety, e.g., for later release. As one example, the agent may be associated with a supramolecular nanostructure (e.g., a supramolecular nanofiber). In some embodiments, the agent may be released from the first peptide moiety and/or the second peptide moiety. As one example, the agent may be released from a supramolecular nanostructure. For example, in FIG. 2B, agent 120 is associated with supramolecular nanostructure 124.

The agent may be associated with the peptide moiety and/or the supramolecular nanostructure by any appropriate method. For instance, in some cases the agent is associated with the peptide moiety and/or the supramolecular nanostructure by mixing the peptide moiety and/or the supramolecular nanostructure in an aqueous solution.

The agent may be released from the supramolecular nanostructure over any appropriate time period. In some embodiments, the agent is released over a period of greater than or equal to 3 hours, greater than or equal to 4 hours, greater than or equal to 8 hours, greater than or equal to 12 hours, greater than or equal to 1 day, greater than or equal to 3 days, greater than or equal to 1 week, or greater. In some embodiments, the agent is released over a period of less than or equal to 4 weeks, less than or equal to 2 weeks, less than or equal to 1 week, less than or equal to 3 days, less than or equal to 1 day, less than or equal to 12 hours, or less. Combinations of these ranges are possible. For example, in some embodiments, the agent is released over a period of greater than or equal to 3 hours and less than or equal to 4 weeks.

The agent may comprise any of a variety of compounds. In some embodiments, the agent comprises an anesthetic. For example, in some embodiments, the agent is an anesthetic. In some embodiments, the agent comprises a sodium channel blocker (e.g., the agent may be a sodium channel blocker). In some embodiments, the anesthetic comprises a sodium channel blocker. The sodium channel blocker may comprise at least one of tetrodotoxin and saxitoxin.

In some embodiments, a supramolecular nanostructure is configured to retain the agent. For example, the supramolecular nanostructure may be configured to retain the agent using sodium channel peptides. The ability of a supramolecular nanostructure to retain an active ingredient may be measured by dialysis. For example, an aqueous solution comprising supramolecular nanostructures may be sealed within dialysis tubing configured to retain the supramolecular nanostructures, while permitting exchange of the agent through the dialysis tubing. By measuring the quantity of the agent released through the dialysis tubing into an external environment (e.g., an external aqueous solution) release of the agent from the supramolecular nanostructures may be measured. The retention of the agent by the supramolecular nano fibers may be determined by the ability of the nano fibers to retain at least 10%, at least 20%, at least 30%, at least 40%, at least 50%, at least 60%, at least 70%, at least 80%, at least 90%, or more of an initially bound agent within the dialysis tubing.

The supramolecular nanostructure may be configured to retain the agent at a temperature. In some embodiments, the supramolecular nanostructure is configured to retain the agent at a temperature of greater than or equal to 0° C., greater than or equal to 10° C., greater than or equal to 20° C., greater than or equal to 30° C., greater than or equal to 35° C., greater than or equal to 37° C., or greater. In some embodiments, the supramolecular nanostructure is configured to retain the agent at a temperature of less than or equal to 80° C., less than or equal to 70° C., less than or equal to 60° C., less than or equal to 50° C., less than or equal to 40° C., less than or equal to 37° C., or less. Combinations of these ranges are possible. For example, in some embodiments, the supramolecular nanostructure is configured to retain the agent at a temperature of greater than or equal to 0° C. and less than or equal to 80° C.

In some embodiments, the supramolecular nanostructure is configured to retain the agent for greater than or equal to 3 hours, greater than or equal to 4 hours, greater than or equal to 8 hours, greater than or equal to 12 hours, greater than or equal to 1 day, greater than or equal to 3 days, greater than or equal to 1 week, or greater. In some embodiments, the supramolecular nanostructure is configured to retain the agent for less than or equal to 4 weeks, less than or equal to 2 weeks, less than or equal to 1 week, less than or equal to 3 days, less than or equal to 1 day, less than or equal to 12 hours, or less. Combinations of these ranges are possible. For example, in some embodiments, the supramolecular nanostructure is configured to retain the agent for greater than or equal to 3 hours and less than or equal to 4 weeks.

Combinations of the forgoing ranges are possible. For example, in some embodiments, the supramolecular nanostructure is configured to retain at least 50% of the agent for greater than or equal to 3 hours when dialyzed at 37° C.

In some embodiments, the supramolecular nanostructure is configured to retain the agent in an environment with a pH greater than or equal to 5, greater than or equal to 5.5, greater than or equal to 6, greater than or equal to 6.5, greater than or equal to 7, greater than or equal to 7.4, greater than or equal to 7.5, or greater. In some embodiments, the supramolecular nanostructure is configured to retain the agent in an environment with a pH less than or equal to 8, less than or equal to 7.5, less than or equal to 7.4, less than or equal to 7, less than or equal to 6.5, less than or equal to 6, less than or equal to 5.5, or less. Combinations of these ranges are possible. For example, in some embodiments, the supramolecular nanostructure is configured to retain the agent in an environment with a pH greater than or equal to 5 and less than or equal to 8.

In some embodiments, the agent is configured to be released in vivo. For example, the agent may be configured to be released within a patient or a subject.

Compositions as described herein may be particularly useful for being administered to a subject in need thereof. In some embodiments, the compositions are used to deliver a pharmaceutically active agent. The composition may be administered in any way known in the art of drug delivery, for example, orally, parenterally, intraocularly, intravenously, intramuscularly, subcutaneously, intradermally, transdermally, intrathecally, submucosally, sublingually, rectally, vaginally, etc.

Once the composition has been prepared, it may be combined with pharmaceutically acceptable excipients to form a pharmaceutical composition. As would be appreciated by one of skill in this art, the excipients may be chosen based on the route of administration as described below, the agent being delivered, and the time course of delivery of the agent. In some embodiments, the pharmaceutical composition comprising a therapeutically effective amount of a composition as described herein, and one or more pharmaceutically acceptable excipients.

Pharmaceutical compositions for use in accordance with the present disclosure may include a pharmaceutically acceptable excipient. As used herein, the term “pharmaceutically acceptable excipient” means a non-toxic, inert solid, semi-solid or liquid filler, diluent, encapsulating material or formulation auxiliary of any type. Some examples of materials which can serve as pharmaceutically acceptable excipients are sugars such as lactose, glucose, and sucrose; starches such as corn starch and potato starch; cellulose and its derivatives such as sodium carboxymethyl cellulose, methylcellulose, hydroxypropylmethylcellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen free water; isotonic saline; citric acid, acetate salts, Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions can be administered to humans and/or to animals, orally, rectally, parenterally, intracisternally, intravaginally, intranasally, intraperitoneally, topically (as by powders, creams, ointments, or drops), bucally, or as an oral or nasal spray.

Liquid dosage forms for oral administration include pharmaceutically acceptable emulsions, microemulsions, solutions, suspensions, syrups, and elixirs. In addition to the active ingredients (i.e., the particles), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3 butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, ethanol, U.S.P., and isotonic sodium chloride solution. In addition, sterile, fixed oils are conventionally employed as a solvent or suspending medium. For this purpose, any bland fixed oil can be employed including synthetic mono or diglycerides. In addition, fatty acids such as oleic acid are used in the preparation of injectables.

The injectable formulations can be sterilized, for example, by filtration through a bacteria retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the inventive particles with suitable non irritating excipients or carriers such as cocoa butter, polyethylene glycol, or a suppository wax which are solid at ambient temperature but liquid at body temperature and therefore melt in the rectum or vaginal cavity and release the microparticles.

Solid dosage forms for oral administration include capsules, tablets, pills, powders, and granules. In such solid dosage forms, the particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of an inventive pharmaceutical composition include ointments, pastes, creams, lotions, gels, powders, solutions, sprays, inhalants, or patches. The particles are admixed under sterile conditions with a pharmaceutically acceptable carrier and any needed preservatives or buffers as may be required. Ophthalmic formulation, ear drops, and eye drops are also contemplated as being within the scope of this disclosure.

The ointments, pastes, creams, and gels may contain, in addition, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the particles in a proper medium. Absorption enhancers can also be used to increase the flux of the compound across the skin. The rate can be controlled by either providing a rate controlling membrane or by dispersing the particles in a polymer matrix or gel.

The present disclosure also provides kits for use in preparing or administering the inventive particles. A kit for forming particles may include the albumin and a dialdehyde as well as any solvents, solutions, buffer agents, acids, bases, salts, targeting agent, etc. needed in the particle formation process. Different kits may be available for different targeting agents. In certain embodiments, the kit includes materials or reagents for purifying, sizing, and/or characterizing the resulting particles. The kit may also include instructions on how to use the materials in the kit. The one or more agents (e.g., pharmaceutically active agent) to be encapsulated in the particle are typically provided by the user of the kit.

Kits are also provided for using or administering the inventive composition or pharmaceutical compositions thereof. The composition (e.g., particles) may be provided in convenient dosage units for administration to a subject. The kit may include multiple dosage units. For example, the kit may include 1-100 dosage units. In certain embodiments, the kit includes a week supply of dosage units, or a month supply of dosage units. In certain embodiments, the kit includes an even longer supply of dosage units. The kits may also include devices for administering the composition or a pharmaceutical composition thereof. Exemplary devices include syringes, spoons, measuring devices, etc. The kit may optionally include instructions for administering the inventive composition (e.g., particles) (e.g., prescribing information).

The term “monomer” as used herein, has its ordinary meaning in the art and may refer to a molecule or a moiety on a molecule that is capable of participating in a reaction to become a part of the essential structure of a polymer.

The term “pendant group” as used herein, refers to a group attached to the backbone of a polymer that is neither oligomeric nor polymeric.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972). The disclosure additionally encompasses compounds as individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic,” as used herein, includes both saturated and unsaturated, nonaromatic, straight chain (i.e., unbranched), branched, acyclic, and cyclic (i.e., carbocyclic) hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Likewise, the term “heteroaliphatic” refers to heteroalkyl, heteroalkenyl, heteroalkynyl, and heterocyclic groups. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In some embodiments, as used herein, “aliphatic” is used to indicate those aliphatic groups (cyclic, acyclic, substituted, unsubstituted, branched or unbranched) having 1-20 carbon atoms. Aliphatic group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The alkyl groups may be optionally substituted, as described more fully below. Examples of alkyl groups include, but are not limited to, methyl, ethyl, propyl, isopropyl, butyl, isobutyl, tert-butyl, 2-ethylhexyl, cyclopropyl, cyclobutyl, cyclopentyl, cyclohexyl, and the like. “Heteroalkyl” groups are alkyl groups wherein at least one atom is a heteroatom (e.g., oxygen, sulfur, nitrogen, phosphorus, etc.), with the remainder of the atoms being carbon atoms. Examples of heteroalkyl groups include, but are not limited to, alkoxy, poly(ethylene glycol)-, alkyl-substituted amino, tetrahydrofuranyl, piperidinyl, morpholinyl, etc.

The terms “alkenyl” and “alkynyl” refer to unsaturated aliphatic groups analogous to the alkyl groups described above, but containing at least one double or triple bond respectively. The “heteroalkenyl” and “heteroalkynyl” refer to alkenyl and alkynyl groups as described herein in which one or more atoms is a heteroatom (e.g., oxygen, nitrogen, sulfur, and the like).

The terms “acyl,” “carboxyl group,” or “carbonyl group” are recognized in the art and can include such moieties as can be represented by the general formula:

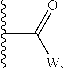

wherein W is H, OH, O-alkyl, O-alkenyl, or a salt thereof. Where W is O-alkyl, the formula represents an “ester.” Where W is OH, the formula represents a “carboxylic acid.” On the other hand, where W is alkyl, the above formula represents a “ketone” group. Where W is hydrogen, the above formula represents an “aldehyde” group.

The term “amide group” is recognized in the art and can include such moieties as can be represented by the general formula:

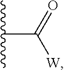

wherein W is NH₂, NH-alkyl, N-dialkyl, NH₃⁺, NH₂⁺-alkyl, NH⁺-dialkyl, or N⁺-trialkyl. As used herein, the carbon atom double-bonded to an oxygen atom of an acyl or amide group is referred to as a “carbonyl carbon”. As used herein, the nitrogen atom of an amide group, bonded to the carbonyl carbon, is referred to as an “amide nitrogen”.

U.S. Provisional Application No. 63/219,790, entitled “Systems and Methods for Delivery Based on Supramolecular Nanostructures,” filed on Jul. 8, 2021, is incorporated herein by reference in its entirety.

The following examples are intended to illustrate certain embodiments of the present disclosure, but do not exemplify the full scope of the disclosure.

### Example 1

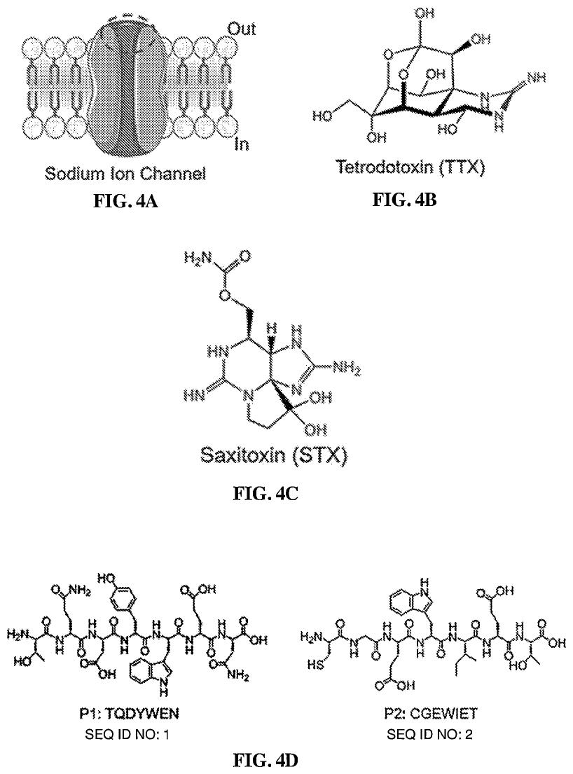

This example describes the selection and initial characterization of site 1 sodium channel blockers (S1SCBs) in solution. S1SCBs block the same sodium channel (e.g., the voltage-gated sodium channel Nav 1.7) as do conventional local anesthetics, but at a different site on the outer surface of the cell. FIG. 4A presents an exemplary sodium ion channel, with the dashed circle indicating site 1. It was hypothesized that S1SCBs such as tetrodotoxin (TTX) and saxitoxin (STX) would have affinity for their specific binding sites on the sodium channel, and that this affinity could be used to create a sustained release system. FIG. 4B presents the chemical structure of TTX, and FIG. 4C presents the chemical structure of STX, according to some embodiments. Two key peptide sequences were selected. The first peptide sequence was TQDYWEN (SEQ ID NO: 1), which, for brevity, is designated throughout the examples herein as P1. FIG. 4D presents the molecular structure associated with these exemplary sequences. The second peptide sequence was CGEWIET (SEQ ID NO: 2), which, for brevity, is designated throughout the examples herein as P2.

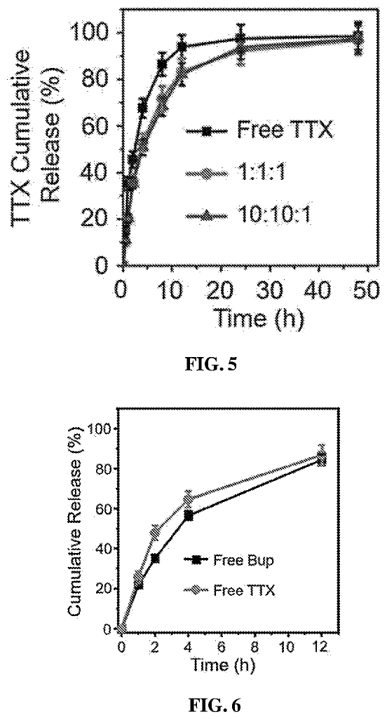

P1 and P2 simultaneously bind S1SCBs at the binding site, and this example examined whether a mixture of the two (termed P1P2) could interact specifically to bind TTX, creating a controlled release system (i.e., slowing its release). Release of TTX from P1P2 from 0.5 mL of P1P2 in dialysis tubing into 14 mL of PBS was quantified by ELISA. FIG. 5 reports these results, indicating the cumulative release of TTX from a solution comprising free TTX sample, a solution wherein P1:P2:TTX=1:1:1 (wherein P1:P2:TTX represents a molar ratio), and a solution wherein P1:P2:TTX=10:10:1, each solution having an identical concentration of 62.6 micromolar TTX.

Ideally, “release” of a free drug should be almost instantaneous. Here, release of free TTX occurred over>12 h, indicating that release of TTX was affected by the specifics of the experimental set-up—a common result in dialysis membrane-based set-ups, which tends to reduce differences in release kinetics between samples. The fact that the release of bupivacaine, another anesthetic, was also slowed and to a very similar degree (shown in FIG. 6) further supports the view that there was interaction between the drugs and the system.

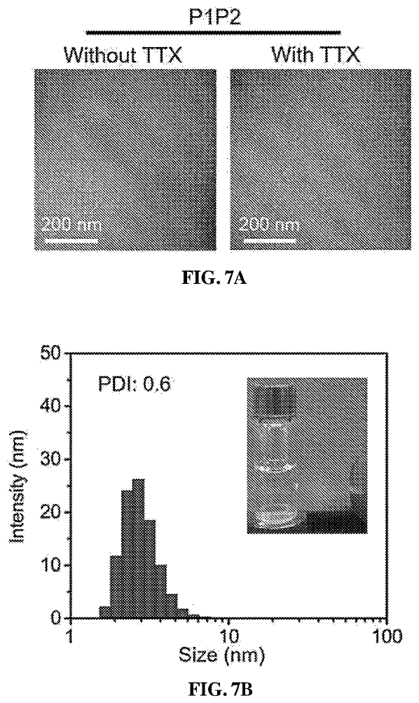

The presence of the dissolved peptides had relatively little effect on the diffusion of TTX, even when the concentrations of P1 and P2 were increased tenfold (FIG. 5). FIG. 7A presents transmission electron microscopy (TEM) images, which showed that no uniform nanostructures formed in P1P2 solutions with or without added TTX. The mean diameter of P1P2 with TTX was 3.0±1.8 nm, and the polydispersity index (PDI) was 0.6, determined by dynamic light scattering (DLS). FIG. 7B presents the size distribution observed within the P1P2 solution by DLS. Moreover, when a 635 nm laser was passed through a solution of P1P2 with TTX, the beam could not be seen (FIG. 7B; this Tyndall phenomenon is indicative of the presence of nanomaterials). These results indicated that the system did not form nanostructures, suggesting that the distances between P1, P2 and TTX were too long to generate effective supramolecular interactions.

### Example 2

This example describes the modification of P1 and P2 (of Example 1) with hydrophobic domains to create peptide moieties (within the examples herein, MPs denote peptide moieties, while MP1 and MP2 denote modifications of P1 and P2 respectively) that assembled into supramolecular nanostructures. For example, referring to FIGS. 2A-2B (described in greater detail above), first peptide moiety 111 could correspond to MP1, second peptide moiety 115 could correspond to MP2, and agent 120 could correspond to TTX, in some embodiments.

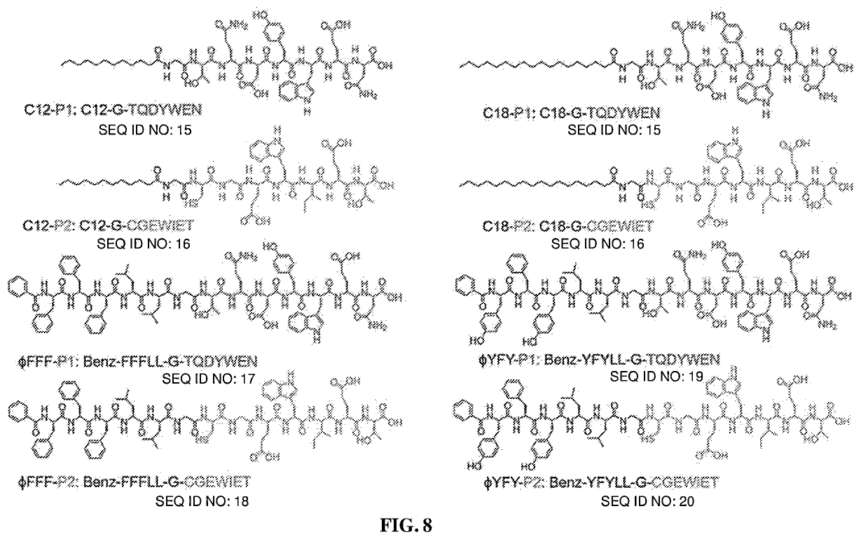

Different peptide moieties, modified with different hydrophobic domains at the N-terminus of P1 and P2 via a glycine (G) linker, were designed. The hydrophobic domains selected were dodecanoic acid (C12), octadecanoic acid (C18), and two peptides, composed of hydrophobic amino acids (F=phenylalanine, Y=tyrosine, L=leucine) and conjugated at the N-terminus to benzoic acid (Benz): Benz-FFFLL (SEQ ID NO: 3) and Benz-YFYLL (SEQ ID NO: 12) (FIG. 8, Table 1, Table 2). The resulting peptide moieties were named C12-Pn (n=1 or 2, corresponding to modification of P1 or P2), C18-Pn, ϕFFF-Pn and ϕYFY-Pn, respectively.

TABLE 1
List of peptides

| Abbreviation | Description | Notes |
|---|---|---|
| P1 | Part of repeat I of Naᵥ1.7 | Peptides with known specific interactionswith |
| P2 | Part of repeat I of Naᵥ1.7 | TTX |
| P1P2 | P1:P2 = 1:1 molar ratio | |
| C12-P1P2 | P1 and P2 with hydrophobic | Amphiphilic peptides that can form |
| | modifications (“modified | nanostructures. |
| C18-P1P2 | peptides”). | From C12 to ϕYFY, the molecular weightof the |
| | | hydrophobic domain increases. |
| ϕFFF-P1P2 | | This synthetic peptide library identified thelead |
| ϕYFY-DP1P2 | | peptide for TTX binding and controlled release. |
| MuQP1P2 | Glu (E) replaced by Gln (Q) in | Known mutant site causing reduced TTX |
| | the P1 and P2 sequences | binding to sodium channel |
| MuDP1P2 | Glu (E) replaced by Asp (D) in | Mutant peptides maintaining the overall |
| | the P1 and P2 sequences | charge characters as P1P2 |
| ScP1P2 | Scrambled P1P2 ScP1: | Mutant peptides that contain the same amino |
| | QDNWTYE (SEQ ID NO: 13) | acids as P1P2 but with scrambled sequence |
| | ScP2: EGWEICT (SEQ ID | |
| | NO: 14) | |
| ϕFFF-MuQP1P2 | Modified MuQP1P2 | Amphiphilic peptides that can form |
| ϕFFF-MuDP1P2 | Modified MuDP1P2 | nanostructures. |
| ϕFFF-ScP1P2 | Modified ScP1P2 | Used to investigate the effect of sequence on the |
| | | specific interaction between TTX and |
| | | nanostructures |

TABLE 2
Peptide names, sequences,
molecular weight (Mw) and purity

| | | | | SEQ |
|---|---|---|---|---|
| | | | Purity | ID |
| Name | Sequence | Mw | (%) | NO: |
| P1 | TQDYWEN | 954.37 | 96.97 | 1 |
| P2 | CGEWIET | 836.34 | 96.63 | 2 |
| C12-P1 | C12-G-TQDYWEN | 1193.56 | 96.17 | 15 |
| C12-P2 | C12-G-CGEWIET | 1075.53 | 96.46 | 16 |
| C18-P1 | C18-G-TQDYWEN | 1277.65 | 97.25 | 15 |
| C18-P2 | C18-G-CGEWIET | 1159.62 | 95.93 | 16 |
| ϕFFF-P1 | Benz-FFFLL-G-TQDYWEN | 1782.79 | 96.76 | 17 |
| ϕFFF-P2 | Benz-FFFLL-G-CGEWIET | 1664.76 | 97.77 | 18 |
| ϕYFY-P1 | Benz-YFYLL-G-TQDYWEN | 1814.78 | 95.37 | 19 |
| ϕYFY-P2 | Benz-YFYLL-G-CGEWIET | 1696.75 | 95.47 | 20 |
| MuQP1 | TQDYWQN | 953.96 | 96.3 | 3 |
| ϕFFF- | Benz-FFFLL-G-TQDYWQN | 1781.81 | 95.45 | 5 |
| MuQP1 | | | | |
| ϕFFF- | Benz-FFFLL-G-CGQWIET | 1663.77 | 95.67 | 6 |
| MuQP2 | | | | |
| MuDP1 | TQDYW [symbol] N | 940.92 | 95.65 | 7 |
| ϕFFF- | Benz-FFFLL-G-TQDYW [symbol] N | 1769.93 | 95.83 | 9 |
| MuDP1 | | | | |
| ϕFFF- | Benz-FFFLL-G-CG [symbol] WIET | 1651.90 | 95.26 | 10 |
| MuDP2 | | | | |
| ϕFFF- | Benz-FFFLL-G-QDNWTYE | 1783.96 | 96.17 | 11 |
| ScP1 | | | | |
| ϕFFF- | Benz-FFFLL-G-EGWEICT | 1665.93 | 95.89 | 21 |
| ScP2 | | | | |
| ϕFFF-G | Benz-FFFLLG-NH₂ | 845.45 | 96.45 | 22 |
| ϕYFY-G | Benz-YFYLLG-NH₂ | 877.44 | 97.24 | 23 |

The purity of all peptides was over 95% as determined by HPLC. Mutant amino acids are highlighted in bold and underlined.

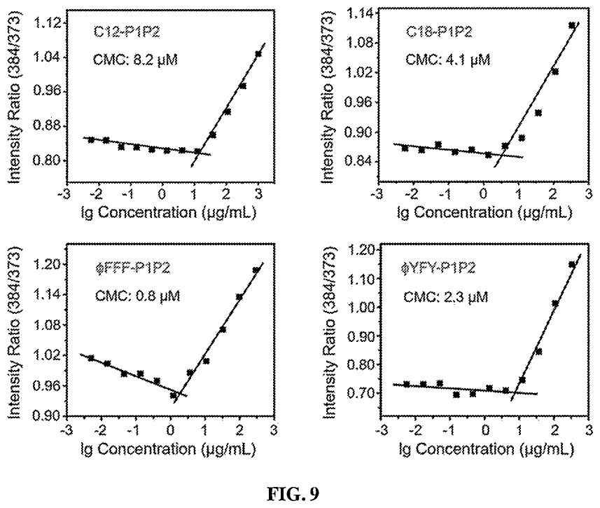

The critical micelle concentration of each peptide moiety was determined. A series of concentrations of C12-P1P2, C18-P1P1, ϕFFF-P1P2, ϕYFY-P1P2, ϕFFF-MuQP1P2, ϕFFF-MuDP1P2 or ϕFFF-ScP1P2 (0 to 200 micromolar) were incubated with a pyrene solution (24 micrograms/L) for 1 h. A fluorescence spectrophotometer (Cary Eclipse Fluorescence Spectrophotometer, Agilent Technologies, Inc. CA, USA) was used to measure the fluorescence intensity of each mixture (excitation at 330 nm). The ratios between the intensities at 384 nm and 373 nm were used to calculate the CMC of the peptide in PBS; the greater the ratio, the more pyrene is encapsulated. The point at which the curve in FIG. 9 begins to rise (i.e. where encapsulation increases) is the CMC.

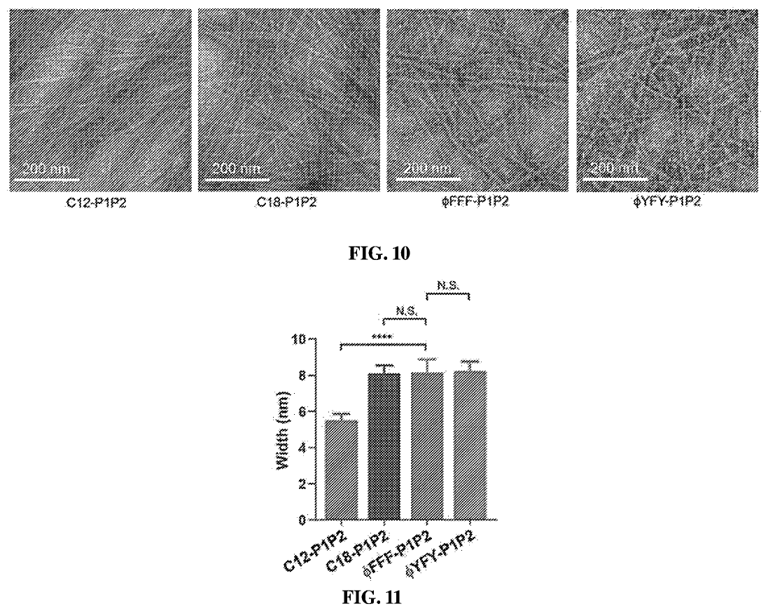

Pairs of peptide moieties (MPs) with the same hydrophobic modification were dissolved in PBS (pH 7.4) in a 1:1 molar ratio (e.g., C18-P1+C18-P2, termed C18-P1P2), both at concentrations of 62.6 micromolar, without TTX. This concentration was higher than their CMC, to ensure that the MPs could form supramolecular nanostructures. FIG. 10 presents TEM images showing that all four MP pairs formed supramolecular nanostructures (more specifically, supramolecular nanofibers). To produce these images, peptide moieties (0.63 micromoles) were dissolved in 5 microliters DMSO and then separated in 1 mL PBS at a series of concentrations, then ultrasonicated at 100 W for 1 min at 25° C. Solutions (1 mL) were placed into a dialysis device (Float-A-LyzerG2 Dialysis Devices, Spectrum Laboratories, Inc.) with a 1000 MW cut-off and dialyzed with 1 L PBS for 48 h to remove DMSO. The resulting solutions were diluted to 62.6 micromolar and characterized by transmission electron microscopy (TEM, Tecnai G2 T20; FEI company, OR, USA) using a negative staining method with uranyl acetate (1.0% w/w).

The average width of supramolecular nanofibers was calculated after measuring the thickness of ten supramolecular nanofibers in TEM images via Imaging J. Samples with other concentrations or components were prepared in the same way. C12-P1P2 nanofibers were an average of 5.6±0.3 nm in width, thinner than C18-P1P2 (8.2±0.4 nm), ϕFFF-P1P2 (8.2±0.6 nm) and ϕYFY-P1P2 (8.3±0.5 nm) (p<0.0001 for each comparison). FIG. 11 presents the average width of each supramolecular nanostructure. No significant difference was observed between the average width of the other supramolecular nanofibers.

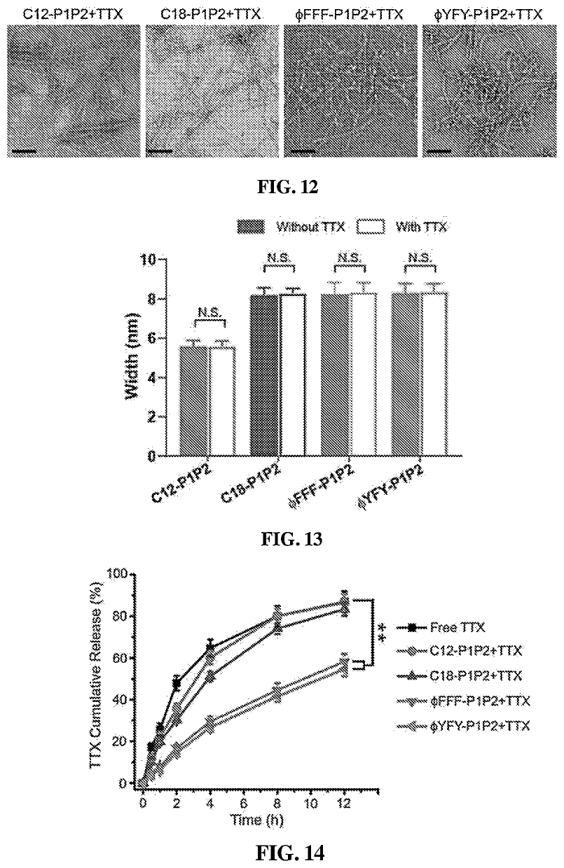

To verify whether the presence of TTX affected the morphology of nanostructures, TEM images of different formulations (MP1:MP2:TTX=1:1:1 molar ratio, 62.6 micromolar) were obtained (FIG. 12). Peptides (0.63 micromoles) were dissolved 5 microliters DMSO and then separated in 1 mL PBS at a series of concentrations, then ultrasonicated at 100 W for 1 min at 25° C. Solutions (1 mL) were placed into a dialysis device (Float-A-LyzerG2 Dialysis Devices, Spectrum Laboratories, Inc.) with a 1000 MW cut-off and dialyzed with 1 L PBS for 48 h to remove DMSO. The resulting solutions were diluted to 62.6 micromolar and characterized by transmission electron microscopy (TEM, Tecnai G2 T20; FEI company, OR, USA) using a negative staining method with uranyl acetate (1.0% w/w). There was no obvious morphological difference between supramolecular nanofibers in the presence of TTX and supramolecular nanofibers in the absence of TTX. The width of supramolecular nanofibers, measured from TEM images, was not affected by the addition of TTX, as shown in FIG. 13.

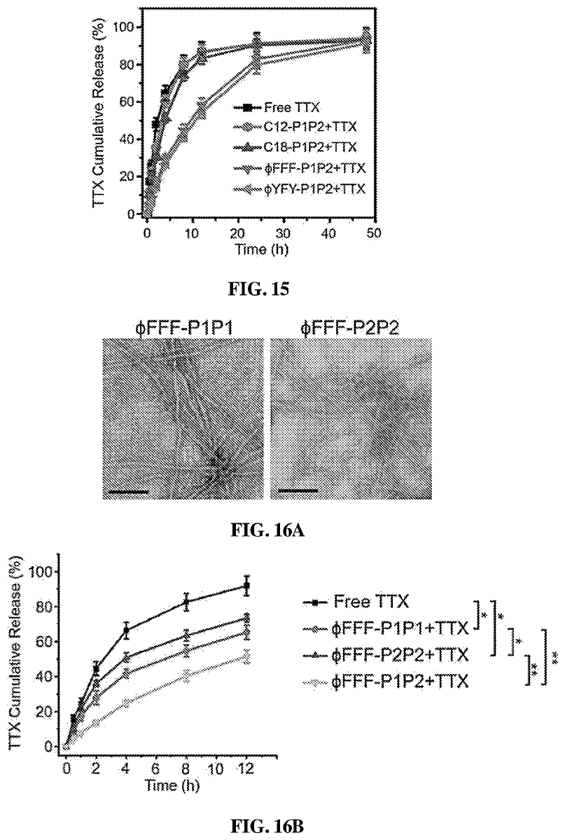

To assess the ability of the supramolecular nanofibers to provide sustained release of TTX, TTX was added to MP mixtures in a 1:1:1 molar ratio (MP1:MP2:TTX), to mimic the ratios at the sodium channel, and release kinetics of TTX from these formulations was evaluated. FIG. 14 shows the release of TTX from each supramolecular nanostructure, as well as the release of free TTX, from the dialysis membrane. FIG. 15 shows the same release profiles as FIG. 14, measured over a longer time-scale. In the C12-P1P2 sample, 86.6±4.1% of TTX was released in 12 h. Similarly, in the C18-P1P2 sample, 83.4±3.2% of TTX was released in 12 h. Release from both the C18-P1P2 sample and the C18-P1P2 sample was similar to the release of free TTX, which was 86.9±5.1% in 12 h. (Four sample measurements were recorded in all samples, and no significant difference between the free TTX sample, the C12-P1P2 sample, and the C18-P1P2 sample). In contrast, ϕFFF-P1P2+TTX and ϕYFY-P1P2+TTX controlled the release of TTX, with 58.3±3.7% and 54.6±3.3% of TTX respectively being released in 12 h.

In this example, and in all examples below, statistical comparisons were performed using the Student t-test (two-sided) unless stated otherwise. Thermal latency, inflammation and myotoxicity scores were reported as medians and quartiles due to their ordinal or non-Gaussian character. Data are presented as means±SD (n=4) in release kinetics, cell work, neurobehavioral, and histology studies. Data were considered statistically significant if p<0.05 (****p<0.0001, ***p<0.001, **p<0.01, *p<0.05); data were considered no significant difference (N.S.) if p>0.05

### Example 3

This example describes the release of TTX from supramolecular nanostructures containing at most one site 1 sodium channel peptide sequence. First, the release kinetics of formulations with either P1 peptide moieties or P2 peptide moieties were investigated. These samples were denoted ϕFFF-P1P1+TTX (similar to ϕFFF-P1P2+TTX, except that all peptide moieties comprised the P1 peptide sequence and not the P2 peptide sequence) and ϕFFF-P2P2+TTX (similar to ϕFFF-P1P2+TTX, except that all peptide moieties comprised the P2 peptide sequence and not the P1 peptide sequence); where the concentrations of peptides and TTX were 125.2 micromolar and 62.6 micromolar respectively.

FIG. 16A presents TEM images collected for each sample, indicating that both ϕFFF-P1P1+TTX and ϕFFF-P2P2+TTX self-assembled into supramolecular nanofibers. FIG. 16B presents the results of the release experiments. ϕFFF-P1P1+TTX released 65.2±4.0% TTX in 12 h, and ϕFFF-P2P2+TTX released 73.4±2.2% in 12 h, faster than release from FFF-P1P2+TTX (52.6±3.5% in 12 h, p<0.01) (FIGS. 16A-16B). These results indicated that the presence of both P1 and P2 sequences was important in nanofiber interactions with TTX.

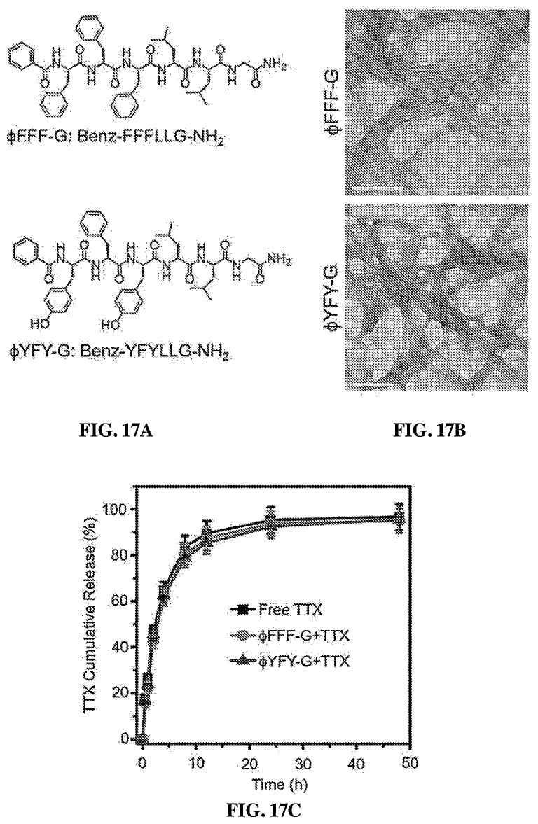

Next, association of TTX with peptide moieties without any sodium channel peptide sequences (e.g., merely comprising glycine, G, connected to a hydrophobic domain) was analyzed as a control experiment. FIG. 17A presents the structure of the peptide moieties ϕFFF-G and YFY-G, formed by conjugating the previously-discussed hydrophobic domains to a glycine residue. FIG. 17B presents TEM images demonstrating that these peptide moieties self-assembled into supramolecular nanofibers. Release experiments were performed as before, and FIG. 17C presents the results. The TTX release kinetics from these peptide moieties were similar to the release kinetics of free TTX.

### Example 4

To investigate interactions between mixtures of peptide moieties, circular dichroism (CD) spectra were obtained for each of P1P2, ϕFFF-P1P2, ϕYFY-P1P2, C12-P1P2, and C18-P1P2, the samples described in previous examples. Circular dichroism (CD) measurements were carried out on a Jasco J-815 CD system (Jasco International Co., Ltd. MD, USA) at 25° C. in phosphate buffer. The signal of 190 nm-260 nm wavelength was collected. Peptides with 20.0 micromolar were measured.

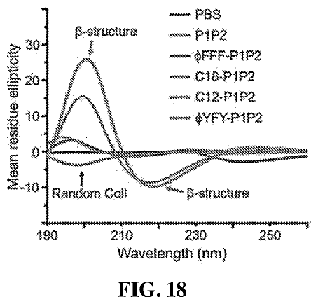

FIG. 18 presents the results of the circular dichroism experiments. Regular beta-structures (such as beta-sheets and beta-turns) form due to continuous hydrogen bonds between peptide backbones. On CD, beta-structures are indicated by a positive peak at approximately 196 nm and a negative peak at 218 nm. P1P2 (20.0 micromolar) yielded a random coil (as indicated by a negative peak at ˜200 nm) (FIG. 18). The introduction of hydrophobic domains produced beta-structures, as evidenced by a positive peak at 200 nm and a negative peak at 218 nm. At 20.0 micromolar concentrations, the positive and negative peaks for beta-structures were the strongest in C12-P1P2, followed by C18-P1P2. The positive peaks at 190-200 nm for ϕFFF-P1P2 and ϕYFY-P1P2 were much weaker, and there were almost no negative peaks at 210-220 nm (FIG. 18). These results indicate that ϕFFF-P1P2 and ϕYFY-P1P2 had lesser degrees of beta-structure than did C12-P1P2 and C18-P1P2, suggesting weaker interaction between their P1 and P2 sequences. Without wishing to be bound by theory, it is believed that the stronger interaction between P1 and P2 in C12-P1P2 and C18-P1P2 might have impeded interaction with TTX, and might explain why these supramolecular nanostructures did not control the release of TTX, while the ϕFFF-P1P2 and ϕYFY-P1P2 supramolecular nanostructures did.

### Example 4

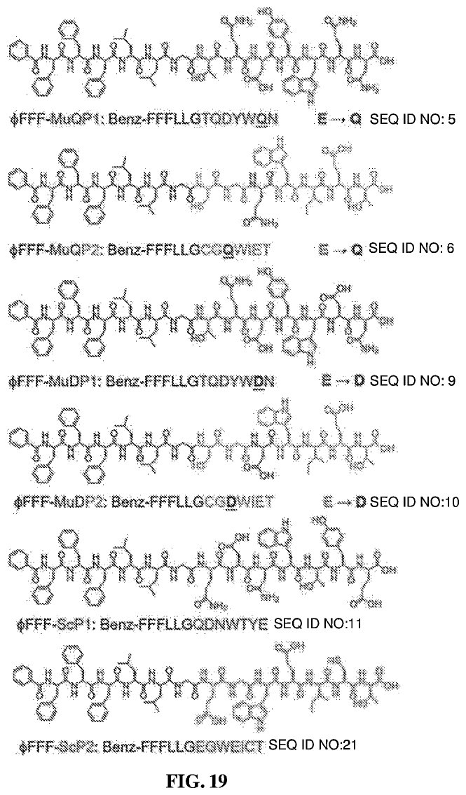

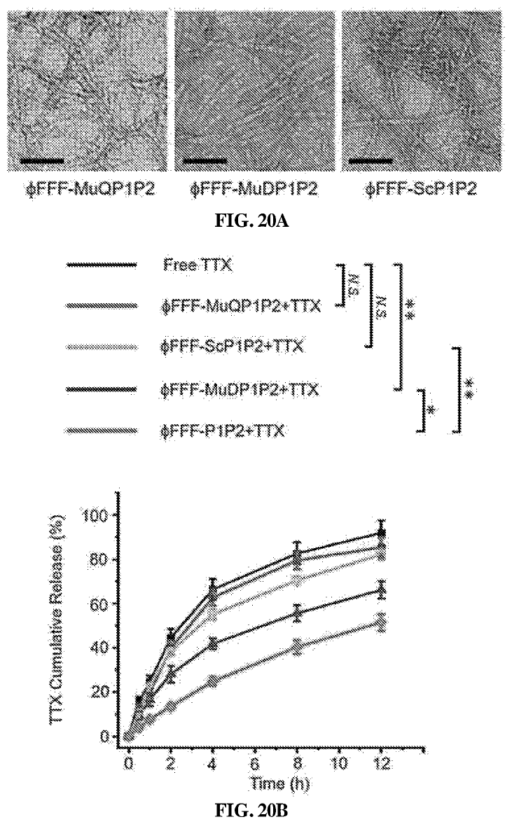

This example demonstrates the effect of mutations of a peptide sequence of a peptide moiety on the release kinetics of the exemplary agent TTX. To study the effect of peptide sequence on TTX release kinetics, MPs were constructed using the TTX-resistant mutant sequences (MuQP1; TQDYWQN (SEQ ID NO: 3)), based on P1 and mutant P2 (MuQP2; CGQWIET (SEQ ID NO: 4), based on P2, to create ϕFFF-MuQP1 (Benz-FFFLLG-TQDYWQN (SEQ ID NO: 5)) and ϕFFF-MuQP2 (Benz-FFFLLG-CGQWIET (SEQ ID NO: 6)) (Table 1, Table 2, FIG. 19). To provide a mutant sequence without a marked change in net charge, one Glu (E) in P1 and P2 was changed to a negatively charged aspartic acid (Asp, D; blue labeled amino acid in FIG. 19), creating mutant sequences MuDP1 (TQDYWDN (SEQ ID NO: 7)) based on P1 and MuDP2 (CGDWIET (SEQ ID NO: 8)) based on P2. Alternative mutations, produced by scrambling the sequences of amino acids in P1 and P2 to form ϕFFF-ScP1(Benz-FFFLLG-QDNWTYE (SEQ ID NO: 11)) and ϕFFF-ScP2 (Benz-FFFLLG-EGWEICT (SEQ ID NO: 6)), respectively. As demonstrated by the TEM micrographs presented in FIG. 20A, ϕFFF-MuQP1P2 (the assembly of ϕFFF-MuP1 and ϕFFF-MuP2), ϕFFF-MuDP1P2 (the assembly of ϕFFF-MuDP1 and ϕFFF-MuDP2), and ϕFFF-ScP1P2 (the assembly of ϕFFF-ScP1 and ϕFFF-ScP2) formed supramolecular nanofibers.

FIG. 20B presents the release of TTX from each sample, as well as release of TTX from free TTX and from ϕFFF-P1P2, as described in greater detail above. None of the mutated peptide moieties slowed the release of TTX to the degree that ϕFFF-P1P2 did. ϕFFF-MuQP1P2, the first mutation, did not slow the release of TTX. ϕFFF-MuDP1P2 slowed the release of TTX (66.1±3.9% released in 12 h) compared to free TTX (91.9±5.6% release; p<0.01). However, TTX release from ϕFFF-MuDP1P2+TTX was still faster than from the non-mutant ϕFFF-P1P2+TTX (51.5±3.7% in 12 h; p<0.01). Additionally, the release of TTX from the scrambled peptide moieties of ϕFFF-ScP1P2 was greatly accelerated relative to ϕFFF-P1P2 (82.1±2.2% release in 12 h; p<0.05 vs. ϕFFF-P1P2+TTX and ϕFFF-MuDP1P2+TTX; no significant difference vs. free TTX and ϕFFF-MuQP1P2+TTX).

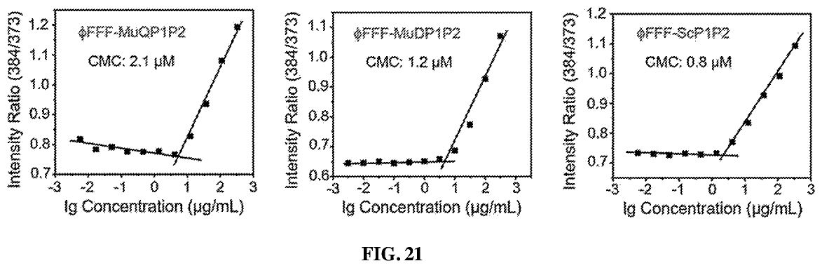

In the TTX release kinetics experiments, the concentrations of all the MP pairs with altered hydrophilic sequences were higher than their CMCs (FIG. 21), i.e., they all formed nanostructures, as shown by TEM (FIG. 10, FIG. 20A). These data suggest that the interaction between TTX and supramolecular nanofibers was sequence-dependent: mutant peptides decreased binding with TTX, and there was none with the scrambled peptides (FIG. 20B).

### Example 5

In this example, a competitive binding assay was performed to further assess the specificity of the interaction between TTX and the MPs. First, TTX solution (0.9 micromolar, 270 ng/mL) was added to each well. Immediately after loading the TTX solution, P1P2, ϕFFF-P1P2, ϕYFY-P1P2, ϕFFF-MuP1P2, ϕFFF-DP1P2, ϕFFF-P1 or ϕFFF-P2 solutions were added into the wells, in the concentrations described in greater detail below. In a control group, the same volume of PBS was added (without the addition of a peptide moiety.

To verify whether the peptides interfered with TAMCP interactions with TTX (aside from the peptides binding TTX itself), the kit was pretreated with 4.3 micromolar ϕFFF-P1P2 or ϕYFY-P1P2 for 30 min—allowing potential block of TAMCP. (4.3 micromolar, was used in some of the above competitive binding experiments. It was above the critical micelle concentration of the modified peptides and was five times higher than the upper limit of the linear detection range of TTX concentrations in the ELISA kit). Known concentrations of TTX (0, 10, 30, 90 and 270 ng/mL) were examined with a TTX ELISA kit pretreated with these peptides, and the results compared to data from kits pretreated with PBS.

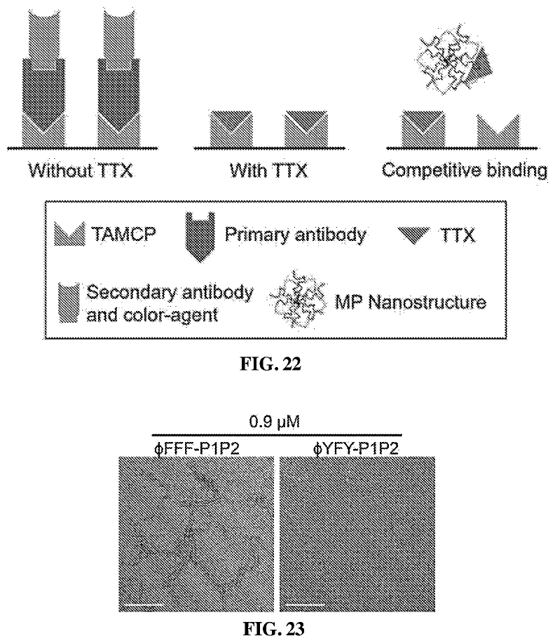

In the TTX ELISA assay (FIG. 22) the bottom of each well was coated with TTX-analogue-modified carrier protein (TAMCP), which can bind TTX specifically but which also acts as an antigen to a primary antibody. If the test sample did not contain TTX, the primary antibody would recognize and bind the protein. The secondary antibody and color agent would then bind to the primary antibody, so that samples without TTX had a dark color. When TTX was present, it specifically bound to the TAMCP, preventing binding of the primary antibody, so that the subsequent reactions could not happen, resulting in a light color. An exemplary schematic illustration of this method is presented in FIG. 22, which illustrates the binding of a color agent to a TAMCP-bound primary antibody in the absence of TTX, but which indicates the displacement of the color agent from the TAMCP in the presence of TTX, which prevents the binding of the primary antibody.

When competitive peptide moiety solutions (such as P1P2, ϕFFF-P1P2 and ϕYFY-P1P2) were added to the ELISA reaction, they bound TTX, allowing more of the TAMCP to capture the color-agent via binding to the primary antibody, as illustrated in FIG. 22. The upper limit of the linear detection range of TTX concentrations in the ELISA kit was 0.9 micromolar (270 ng/mL, from the instructions in the ELISA kit). To evaluate the competitive binding capability of peptides, 0.9 micromolar peptide, MP, or TTX solutions (P1P2, ϕFFF-P1P2 and YFY-P1P2) were used (TTX=270 ng/mL).

TABLE 3
Competitive binding of TTX by peptides as determined by TTX ELISA

| | | Concentration | Percentage | Percentage |
|---|---|---|---|---|
| | | of TTX | of TTX | of TTX |
| Molar Ratios of | | detected | detected | bound by peptide |
| Components ^{a} | Groups | (ng/mL) | (%) | (%) |
| 0:0:1 | Without peptide | 270.0 ± 3.2 | 100.0 ± 1.2 | 0.0 ± 1.2 |
| 1:1:1 | P1P2 | 252.9 ± 8.2 | 93.7 ± 3.0 | 6.3 ± 3.0 |
| | ϕFFF-P1P2 | 92.8 ± 13.2 | 34.4 ± 4.9 | 65.6 ± 4.9 |
| | ϕYFY-P1P2 | 236.2 ± 9.5 | 87.5 ± 3.5 | 12.5 ± 3.5 |
| 5:5:1 | P1P2 | 242.7 ± 4.8 | 89.9 ± 1.8 | 10.1 ± 1.8 |
| | ϕFFF-P1P2 | 15.4 ± 6.8 | 5.7 ± 2.5 | 94.3 ± 2.5 |
| | ϕYFY-P1P2 | 21.2 ± 9.1 | 7.8 ± 3.4 | 92.2 ± 3.4 |
| | ϕFFF-MuDP1P2 | 69.9 ± 11.7 | 25.9 ± 4.3 | 74.1 ± 4.3 |
| | ϕFFF-MuQP1P2 | 233.6 ± 10.3 | 86.5 ± 3.8 | 13.5 ± 3.8 |
| 10:1 | ϕFFF-P1P1 | 72.1 ± 12.2 | 26.7 ± 4.5 | 73.3 ± 4.5 |
| | ϕFFF-P2P2 | 102.2 ± 9.8 | 37.8 ± 3.6 | 62.2 ± 3.6 |

In all groups, the concentration of TTX added was 270 ng/mL (0.9 μM). n = 4, data are means ± SD.
^{a} Ratio of P1-containing peptide:P2-containing peptide:TTX or peptide:TTX

As shown in Table 3, when P1 and P2 were added to TTX at a molar ratio of 1:1:1 (P1:P2:TTX), the TTX concentration detected by ELISA was 93.7±3.0% of the concentration measured without peptides, i.e. 6.3±3.0% of TTX was bound by P1P2 (n=4, p<0.05 vs. samples without peptides; ratio=0:0:1)).

With addition of ϕFFF-P1P2 at the same ratio, 65.6±4.9% of TTX was bound (n=4, p<0.01 vs. samples without peptides). At the same molar ratio, ϕYFY-P1P2 bound 12.5±3.5% of TTX (n=4, p<0.01 vs. samples without peptides), which may be because 0.9 micromolar was below the CMC of ϕYFY-P1P2 which therefore could not form nanostructures (FIG. 23). The absence of supramolecular nanostructures in the sample comprising ϕYFY-P1P2 was confirmed by TEM microscopy, shown in FIG. 23, for both ϕYFY-P1P2 and ϕFFF-P1P2.

When the molar ratio of ϕFFF-P1P2 or ϕYFY-P1P2 to TTX was increased to MP1:MP2:TTX=5:5:1 (MP concentration: 4.3 micromolar), where the concentrations of both ϕFFF-P1P2 and ϕYFY-P1P2 were greater than their CMCs, they bound 94.3±2.5% and 92.2±3.4% of TTX respectively (n=4, p<0.01 vs. samples without peptides) (Table 3, FIG. 9). In contrast, increasing the molar ratio of unmodified P1 and P2 to TTX to 5:5:1 did not significantly increase binding of TTX (n=4, no significant difference vs. 1:1:1 group).

Modified peptides with only P1 or P2, ϕFFF-P1P1 and ϕFFF-P2P2, at 8.6 micromolar (twice the molarity of MPs of other MP pairs since there is only one peptide) bound 73.3±4.5% and 62.2±3.6% of TTX, respectively, which was less than in the ϕFFF-P1P2 group (p<0.01).

The modified mutant peptide 4.3 micromolar ϕFFF-MuDP1P2 bound more TTX than did 4.3 micromolar ϕFFF-MuQP1P2 (74.1±4.3% vs. 13.5±3.8%, p<0.01), suggesting that preserving the charge of the native sequence is important to binding of TTX. However, both mutants bound less TTX than did ϕFFF-P1P2 (p<0.01) (Table 3). These results suggested that ϕFFF-P1P2 had the best binding to TTX, and therefore ϕFFF-P1P2 was used in the following study.

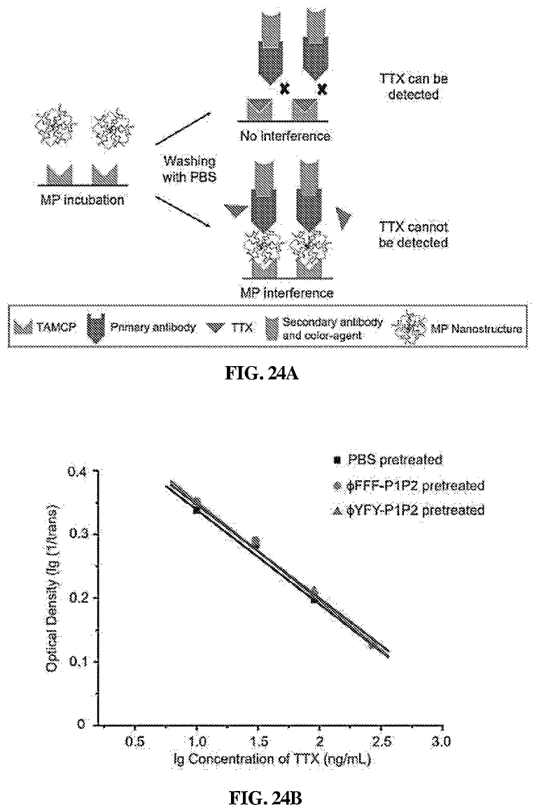

The competitive binding assay was not affected by potential interactions between ϕFFF-P1P2 or ϕYFY-P1P2 and TAMCP. FIG. 24A schematizes the potential interaction between the peptide moieties (MPs) and TAMCP, illustrating how potential interactions could reduce measured intensity. However, the sensitivity and linearity of TTX detection by the ELISA kit was not affected by pretreatment with ϕFFF-P1P2 or ϕYFY-P1P2. This is demonstrated in FIG. 24B, which shows the optical density of a color agent observed as a function of TTX concentration, with and without ϕFFF-P1P2 or ϕYFY-P1P2.

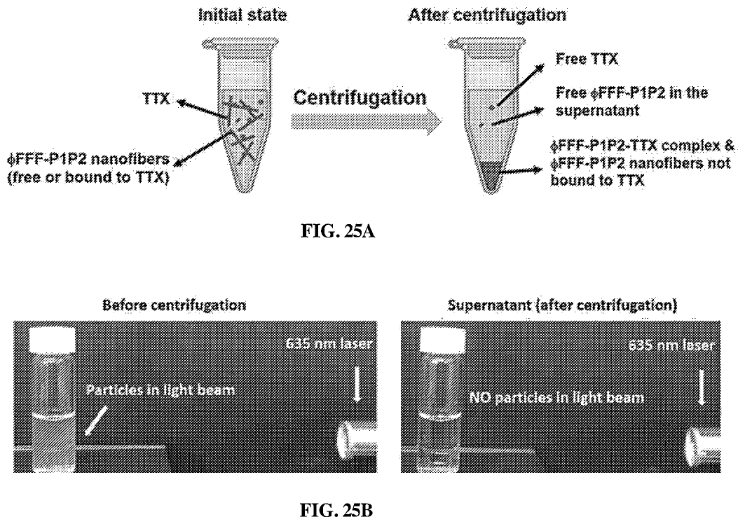

A method was designed for the calculation of Kd between supramolecular nanofibers and TTX. In brief, formulations containing 20 μM peptide and 20 μM TTX were centrifuged at 15,000 rpm for 5 min. FIGS. 25A-25B illustrate this process schematically (FIG. 25A) and in photographs (FIG. 25B). The concentration of free peptide and free TTX in the supernatant were quantified by HPLC and ELISA, respectively. Only 0.21 μM free peptide (1% of the total peptides) could be detected in the supernatant after centrifugation. The peptide in the supernatant could not form nanostructures since its concentration was below the CMC.

More precisely, to determine Kd, 0.5 mL 20 micromolar ϕFFF-P1P2 or ϕFFF-DP1P2, and 20 micromolar TTX was prepared in PBS and placed at room temperature (25° C.) for 30 min. The solution was centrifuged. The centrifugation speed was gradually increased to 15,000 rpm and kept at this speed for 5 min. The concentration of ϕFFF-P1P2 or ϕFFF-DP1P2 in the supernatant was analyzed by HPLC, and the concentration of TTX in the supernatant was measured by TTX ELISA Kit.

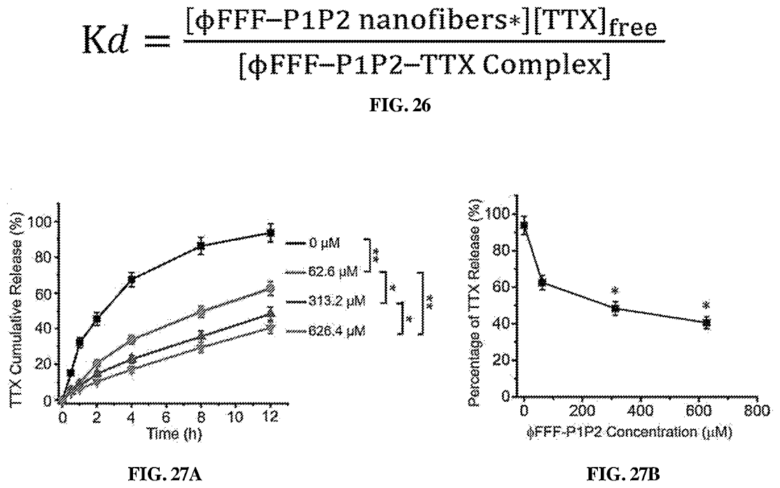

The Kd between ϕFFF-P1P2 nanofiber and TTX was determined by formula (1), also presented in FIG. 26:

$$Kd = \frac{{[ {}_{ϕ} FFF - P 1 P 2 nanofibers * ] [ TTx ]}_{free}}{[ {}_{ϕ} FFF - {P}_{1} {P}_{2} - TTX Complex ]} \qquad ( 1 )$$

where ‘nanofibers*’ refers to supramolecular nanofibers not bound to TTX, and where the bracket notation indicates a molar concentration of a species. In the context of this equation, the subscript ‘free’ refers to a concentration measured in the supernatant. The concentration [ϕFFF-P1P2-TTX complex] was determined by the equation [ϕFFF-P1P2-TTX complex]=[TTX]ᵢₙᵢₜᵢₐₗ-[TTX]_{free}, and the concentration [ϕFFF-P1P2 nanofibers*] was determined by the equation [ϕFFF-P1P2 nanofibers*]=[ϕFFF-P1P2]ᵢₙᵢₜᵢₐₗ-[IFFF-P1P2]_{free}-[ϕFFF-P1P2-TTX complex].

The Tyndall effect (TE) of the resultant solution was tested using a 635 nm red laser pointer, in order to determine whether nanoscale structure was present. The TE image of the solution was recorded photographically. The solution was then centrifuged at 15,000 rpm for 5 min and the resultant supernatant was illuminated with the same 635 nm red laser pointer. The TE image of the supernatant was also recorded. If particles in suspension were nanoscale and were smaller than the wavelength of visible light (˜400 to 760 nm), then a beam of light through the solution would have been scattered. The Tyndall effect was clearly observed with a 635 nm laser passing through a ϕFFF-P1P2+TTX solution, indicating the presence of nanostructures. The absence of Tyndall effect in the supernatant (FIGS. 25A-25B) further confirmed the absence of nanostructures in the supernatant.

The concentration of TTX in the supernatant was 0.34 micromolar. It should be noted that free TTX cannot be centrifuged to the bottom of the tube, as evidenced by the fact that a) TTX can exist in stable solution at much higher concentrations, b) if a TTX solution is centrifugated, the concentration of free TTX afterwards was essentially identical to the initial TTX concentration (19.98 vs. 20 micromolar; Table 4). These results confirmed that only TTX bound to supramolecular nanofibers could be centrifuged to the bottom of the tube.

TABLE 4
Determination of Kd of ϕFFF-P1P2 nanofibers
a nd ϕFFF-MuDP1P2 nanofibers with TTX^{a}

| | Peptide remaining^{b} | TTX remaining^{b} | |
|---|---|---|---|
| | in the | in the | |
| | supernatant | supernatant | Kd |
| Group | (μM) | (μM) | (nM) |
| Free TTX | — | 19.98 ± 0.05 | — |
| ϕFFF-P1P2 | 0.21 ± 0.01 | 0.34 ± 0.02 | 2.2 ± 0.3 |
| ϕFFF-MuDP1P2 | 0.29 ± 0.02 | 2.84 ± 0.04 | 372.4 ± 67.5 |

^{a}The initial concentrations of TTX, ϕFFF-P1P2, and ϕFFF-MuDP1P2 were 20 μM. In free TTX group, no peptides were added. Data are means ± SD; n = 4.
^{b}After binding and centrifugation.

The Kds of ϕFFF-P1P2 and ϕFFF-MuDP1P2 with TTX calculated by this approach were 2.2±0.3 nM and 372.4±67.5 nM (Table 4), respectively. The proposed approach and calculated Kd was not intended to be used for direct comparison to the binding of TTX to the natural sodium channel proteins (Kd≈1.3 nM), but to allow comparison of the binding of TTX by different formulations. Upon mixing, TTX was adsorbed by the supramolecular nanofibers so that only 0.34 micromolar TTX could be detected in the supernatant after the centrifugation of a solution containing 20 micromolar ϕFFF-P1P2 and 20 micromolar TTX, indicating that 98% of TTX was taken up by ϕFFF-P1P2 supramolecular nanofibers Since the two solutions were equimolar, this corresponded to an efficiency of 98% on a molar basis.

### Example 6

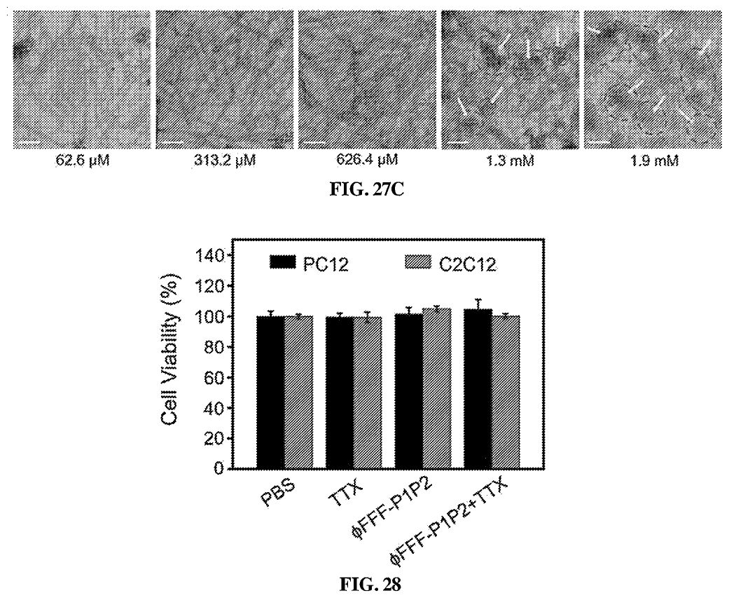

Increasing the ratios of peptide to TTX above the 1:1:1 used in the preceding release experiments was observed to further control the release of TTX, in a manner analogous to increasing the proportion of polymer to drug in other systems. At a constant TTX concentration of 62.6 micromolar, release kinetics were assessed at molar ratios (ϕFFF-P1: ϕFFF-P2:TTX) of 5:5:1 and 10:10:1 (n=4), with corresponding ϕFFF-P1P2 concentrations of 313.2 micromolar and 626.4 micromolar (FIG. 27A). The results are reported in FIGS. 27A-27B. FIG. 27A presents the time-release profile at each molar ratio, represented in terms of an overall molar concentration of each peptide moiety. FIG. 27B presents the total percentage of the TTX released after 12 h, as a function of peptide moiety concentration. Release at the 5:5:1 ratio (with 313.2 micromolar ϕFFF-P1P2) was slower than in the 1:1:1 group (with 62.6 micromolar ϕFFF-P1P2) (p<0.05, at 12 h time point), and was slower yet at the 10:10:1 ratio (626.4 micromolar; p<0.05 vs. 5:5:1 group, at the 12 h time point) (FIG. 27B). Further increasing the ϕFFF-P1: ϕFFF-P2:TTX ratio (i.e. 20:20:1, 1.3 mM and 30:30:1, 1.9 mM) resulted in the formation of precipitate in the solution. FIG. 27C presents evidence for the formation of a precipitate, showing TEM images of each solution, and indicating the presence of aggregates using dashed arrows and circles. Consequently, 626.4 micromolar ϕFFF-P1P2 (which provided the best control of TTX release) was selected for toxicity experiments.

### Example 7

In this example, cytotoxicity of formulations was tested in vitro in myotubes from the myoblast C2C12 cell line to assess potential myotoxicity, and the pheochromocytoma PC12 cell line to assess potential neurotoxicity.

Cell culture of C2C12 mouse myoblasts (American Type Culture Collection (ATCC) CRL-1772) and PC12 rat adrenal gland pheochromocytoma cells (ATCC, CRL-1772) was performed as reported. In brief, C2C12 cells were cultured in DMEM with 10% FBS and 1% Penicillin Streptomycin (Invitrogen). Cells were seeded onto a 24-well plate at 50,000 cells/mL and incubated for 10-14 days in DMEM with 1% FBS and 1% Penicillin Streptomycin to differentiate into myotubules. PC12 cells were grown in DMEM with 12.5% horse serum, 2.5% FBS and 1% Penicillin Streptomycin. Cells were seeded onto a 24 well-plate, and 50 ng/mL nerve growth factor (Invitrogen) was added 24 h after seeding.

To determine the cytotoxicity of the formulations, cells were exposed to TTX, ϕFFF-P1P2 and ϕFFF-P1P2+TTX (TTX concentration: 62.6 micromolar; ϕFFF-P1P2 concentration: 626.4 micromolar) using a 24-well Transwell® membrane system (Costar 3495, pore size 0.4 micrometer) (Corning Incorporated, ME, USA). Cells were incubated in 0.9 mL of media in the cell culture wells, and 100 microliters of test samples were added above the Transwell® membranes, which were immersed in the media in the wells. Cell viability was evaluated by the MTS assay (Promega, WI, USA) 96 h after incubation.

After 96 hours of incubation with test substances (PBS, TTX, ϕFFF-P1P2 and ϕFFF-P1P2+TTX; TTX concentration: 62.6 micromolar; ϕFFF-P1P2 concentration: 626.4 micromolar), cell viability by MTS assay was >95% in all groups (FIG. 28) (n=4, no significant difference: all groups vs. PBS group).

### Example 8

The ability of the formulations to extend the duration of nerve block in vivo was evaluated in a rat model of sciatic nerve block. In brief, rats (n=4 in each group) were injected at the left sciatic nerve with 0.2 mL of formulations (Table 5). They then underwent neurobehavioral testing to determine the duration of sensory and motor nerve blockade in both hind paws.

TABLE 5
Efficacy of TTX formulations in sciatic nerve block.

| | | | Sensory | Motor | |
|---|---|---|---|---|---|
| | TTX | Mortality | Block Time | Block Time | P |
| Groups | (μM) | (%) | (h) | (h) | Value^{a} |
| Free TTX | 15.7 | 0 | 0 | 0 | — |
| | 31.3 | 0 | 0 | 0 | — |
| | 47.0 | 0 | 1.1 ± 1.2 | 1.6 ± 1.3 | >0.05 |
| | 62.6 | 0 | 2.2 ± 0.5 | 2.7 ± 0.6 | >0.05 |
| | 78.3 | 100 | — | — | — |
| ϕFFF-P1P2 + TTX | 15.7 | 0 | 0.8 ± 0.6 | 1.2 ± 0.7 | >0.05 |
| | 31.3 | 0 | 4.7 ± 0.8 | 5.5 ± 0.6 | >0.05 |
| | 47.0 | 0 | 6.1 ± 1.1 | 7.3 ± 0.6 | >0.05 |
| | 62.6 | 0 | 7.9 ± 0.7 | 9.5 ± 0.9 | <0.05 |
| | 78.3 | 0 | 10.8 ± 0.7 | 12.7 ± 0.9 | <0.05 |
| | 94.0 | 0 | 13.2 ± 1.0 | 15.3 ± 1.2 | <0.05 |
| | 109.6 | 0 | 15.9 ± 1.3 | 18.1 ± 2.2 | <0.05 |
| | 125.3 | 100 | — | — | — |
| P1P2 + TTX | 47.0 | 0 | 1.4 ± 1.0 | 1.9 ± 1.3 | >0.05 |
| | 62.6 | 0 | 2.1 ± 0.5 | 2.8 ± 0.3 | >0.05 |
| ϕFFF-MuQP1P2 + TTX | 62.6 | 0 | 2.4 ± 0.5 | 3.1 ± 0.6 | >0.05 |
| ϕFFF-MuDP1P2 + TTX | 62.6 | 0 | 3.6 ± 0.6 | 4.2 ± 0.7 | >0.05 |
| PBS | 0 | 0 | 0 | 0 | — |
| ϕFFF-P1P2 | 0 | 0 | 0 | 0 | — |

^{a}For the comparison of the durations of sensory and motor block. Data are means ± SD; n = 4.

Animal studies were conducted following protocols approved by the Boston Children's Hospital Animal Care and Use Committee in accordance with the guidelines of the International Association for the Study of Pain. Adult male Sprague-Dawley rats (Charles River Laboratories) weighing 350-400 g were housed in groups under a 12-h/12-h light/dark cycle with lights on at 6:00 AM.

After being anesthetized with isoflurane-oxygen, the animals were injected with 0.2 mL of each formulation using a 23-G needle. The needle was introduced posteromedial to the greater trochanter, pointing in the anteromedial direction, and upon contact with bone the TTX formulations were injected onto the sciatic nerve.

Sensory nerve block was examined at predetermined time points by a modified hotplate test (hind-paw thermal latency). The plantar surface of the rat's hind paw was placed on a preheated hot plate at 56° C. The time until the animal withdrew its foot (the thermal latency) was recorded. Animals that did not retract the foot after 12 s were removed from the hotplate to prevent thermal injury. A thermal latency above 7 s was considered a successful nerve block for the purpose of calculating the duration of nerve block. Measurements were repeated three times at each time interval.

Motor nerve block was assessed by a weight-bearing test to determine the motor strength of the rat's hind paw, as described previously. In brief, the rat was positioned with one hind paw on a digital balance and was allowed to bear its own weight. The maximum weight that the rat could bear without the ankle touching the balance was recorded, and motor block was considered achieved when the motor strength was less than half-maximal, as described previously.

Drug release studies were performed by placing 500 microliters of TTX-loaded peptides into a Slide-A-Lyzer MINI dialysis device (Thermo Scientific, Tewksbury, MA) with a 3,500 MW cut-off, and dialyzing against 14 mL of PBS at 37° C. on a platform shaker (New Brunswick Innova 40, Eppendorf North America, NY, USA) at a speed of 200 rpm. At predetermined intervals, the dialysis solution was exchanged with fresh, prewarmed (37° C.) PBS. The concentration of TTX was quantified by ELISA following the instruction. Reagents were provided in a kit. In brief, 50 microliters of samples were added to each well and incubated for 30 min at 25° C., and then 50 microliters of primary antibody was added and incubated for another 30 min at 25° C. The solution in each well was removed, and the well was washed four times with 300 microliters 1× washing buffer. 100 microliters of enzyme-conjugated secondary antibody was added to each well and incubated for 30 min at 25° C., then the well was washed four times with 300 microliters 1× washing buffer. 100 microliters of color solution was added and incubated for 15 min at 25° C., followed then 50 microliters of stop solution were added. The optical density (OD) at 450 nm of the solution was tested.

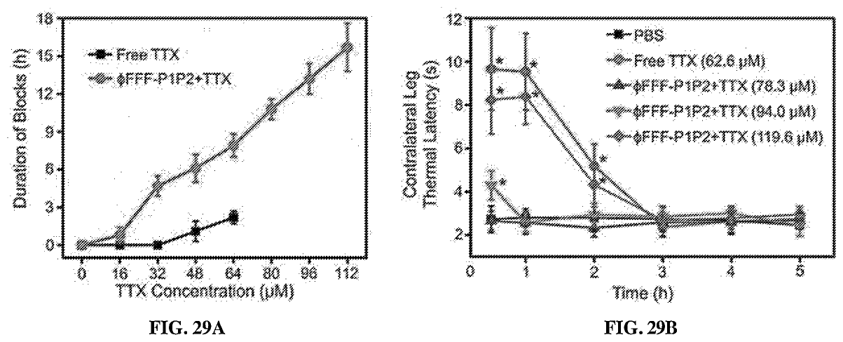

Deficits on the injected (left) side reflected nerve block; deficits on the uninjected right (contralateral) side reflected systemic TTX distribution. FIG. 29A presents exemplary data on the duration of sensory nerve blocks. Data are means±SD; n=4. FIG. 29B presents Contralateral Leg Thermal Latency as a function of time following the injection. Table 5 reports the efficacy of the TTX formulations in the sciatic nerve, as well as the mortality of each dose. The duration of successful nerve block from TTX alone increased with increasing dose, but toxicity became dose limiting (FIG. 29B, Table 5). 62.6 micromolar TTX generated significant contralateral block (FIG. 29B), indicating severe systemic toxicity, while 78.3 micromolar TTX was uniformly fatal (Table 5). Delivery with unmodified peptides (P1P2+TTX), or peptides with Glu changed to Gln (ϕFFF-MuQP1P2+TTX), did not improve nerve block or toxicity (no significant difference: P1P2+TTX and ϕFFF-MuQP1P2+TTX vs. free TTX) (Table 5).

ϕFFF-MuDP1P2+TTX could prolonged the duration of nerve block to a certain degree. For example, 62.6 micromolar free TTX provided sensory nerve block lasting 2.2±0.5 h; block from the same concentration of TTX in ϕFFF-MuDP1P2+TTX lasted 3.6±0.6 h (Table 5). However, when TTX was delivered in ϕFFF-P1P2+TTX, the duration of nerve block was markedly prolonged. For example, nerve block from 62.6 μM TTX in FFF-P1P2+TTX lasted 3.6-fold longer (7.9±0.7 h; p<0.001) than that of free TTX. The longest sensory block, with 109.6 μM TTX, was 15.9±1.3 h.

Systemic toxicity of TTX was decreased by delivery in ϕFFF-P1P2+TTX. There were no deficits in the contralateral (uninjected) extremities at TTX concentrations ≤78.3 μM in ϕFFF-P1P2+TTX (FIG. 29B, Table 5). 94.0 μM TTX in ϕFFF-P1P2+TTX caused increased contralateral thermal latency (4.3±0.7 s) compared to animals injected with phosphate buffered saline (2.7±0.6 s) (n=4, p<0.01), and 109.6 μM TTX in ϕFFF-P1P2+TTX caused contralateral latency (˜8 s) comparable to that seen with 62.6 μM free TTX (FIG. 29B). In general, the durations of sensory and motor block were similar (Table 5) with motor block being ≤15% longer, especially at higher concentrations of TTX. The peptides (ϕFFF-P1P2) alone did not produce deficits in the injected or contralateral extremity (Table X5).

Animals were euthanized by carbon dioxide at 4 d or 14 d after formulation administration. Four days and 14 days after injection of each formulation, the sciatic nerves and adjacent tissues were harvested, sectioned, stained with hematoxylin-eosin (H&E), and tissue reaction (inflammation and myotoxicity) was assessed. The sciatic nerve and surrounding tissue were harvested and underwent standard procedures to produce H&E-stained slides. The samples were scored for inflammation (0-4) and myotoxicity (0-6), as reported. All scoring and other histological assessments were performed by an observer (M. M.) blinded as to the nature of the individual samples. The inflammation score was a subjective quantification of severity in which 0 was normal and 4 was severe inflammation. The myotoxicity score was determined by the nuclear internalization and regeneration of myocytes, two representative characteristics of local anesthetics' myotoxicity. Nuclear internalization was characterized by myocytes having nuclei located away from their usual location at the periphery of the cell. Regeneration was characterized by the presence of shrunken myocytes with basophilic cytoplasm. The scoring scale was as follows: 0=normal; 1=perifascicular internalization; 2=deep internalization (more than five cell layers); 3=perifascicular regeneration; 4=deep tissue regeneration (more than five cell layers); 5=hemifascicular regeneration; 6=holofascicular regeneration.

To evaluate the neurotoxicity of formulations, the sciatic nerve samples were fixed in Karnovsky's KII Solution (2.5% glutaraldehyde, 2.0% paraformaldehyde, 0.025% calcium chloride in 0.1 M cacodylate buffer, pH 7.4). Samples were treated with osmium tetroxide for postfixation and subsequently were stained with uranyl acetate, were dehydrated in graded ethanol solutions, and were infiltrated with propylene oxide/TAAB 812 Resin (TAAB Laboratories, Calleva Park, United Kingdom) mixtures. Since hematoxylin-eosin staining is not very sensitive for nerve injury, nerves were prepared for staining with toluidine blue and TEM. Tissue sections of a 0.5 micrometer thickness were stained with toluidine blue, followed by high-resolution light microscopy.

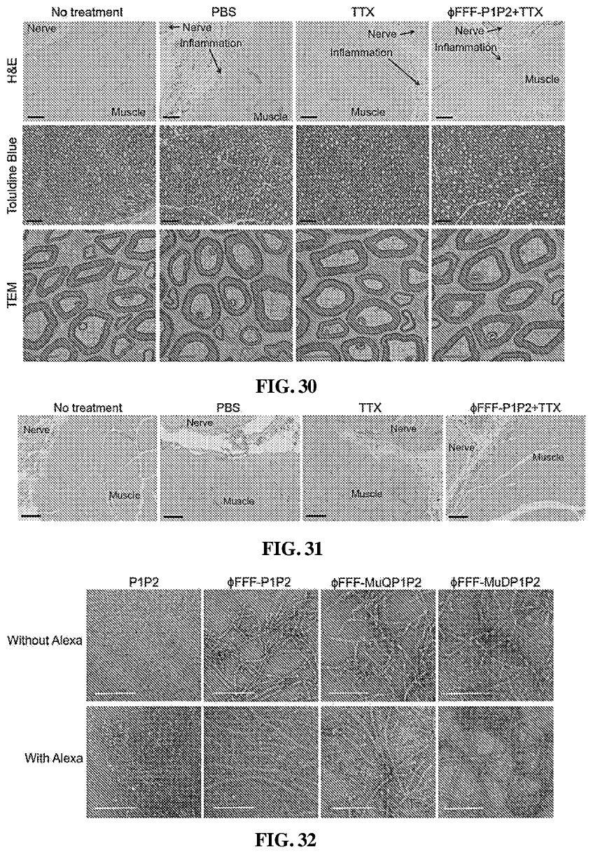

FIG. 30 presents micrographs of hematoxylin-eosin and toluidine blue, as well as TEM micrographs, of tissue sections collected 4 days after injection. Similarly, FIG. 31 presents images of tissue sections collected 14 days after injection. Table 6 presents the quantified myotoxicity and inflammation resulting from this analysis. There was no statistically significant difference in myotoxicity or inflammation scores between any group and untreated animals at either time point. There was no difference between any group and untreated animals.

TABLE 6
Myotoxicity and inflammation

| | | Myotoxicity | Myotoxicity | Inflammation | | Inflammation |
|---|---|---|---|---|---|---|
| | TTX | Score (range) | Score (range) | Score (range) | | Score (range) |
| Formulation | (μM) | Day 4 | Day 14 | Day 4 | | Day 14 |
| No treatment | 0 | 0 (0-1.0) | 0 (0-1.0) | 0 | (0-1.0) | 0 (0-1.0) |
| PBS | 0 | 0 (0-1.0) | 0 (0-1.0) | 0.5 | (0-1.0) | 0 (0-1.0) |
| P value | | >0.05 | >0.05 | >0.05 | | >0.05 |
| Free TTX | 62.6 | 0 (0-1.0) | 0 (0-1.0) | 0.5 | (0-1.0) | 0 (0-1.0) |
| P value | | >0.05 | >0.05 | >0.05 | | >0.05 |
| ϕFFF-P1P2 + TTX ^{a} | 94.0 | 0 (0-1.0) | 0 (0-1.0) | 0.6 | (0-1.0) | 0 (0-1.0) |

| | | | | |
|---|---|---|---|---|
| P value | >0.05 | >0.05 | >0.05 | >0.05 |

^{a} The ϕFFF-P1P2 dose was 0.9 mg/rat.
Inflammation scores: 0-4; myotoxicity scores: 0-6. Data are medians with 25^{th} and 75^{th} percentiles in parentheses.
P values are for the comparison of the tissue reaction of test compounds to that of the untreated group. Data are means ± SD; n = 4.

### Example 9

In this example, the near infrared dye Alexa647 was covalently conjugated to 1% of the unmodified peptides to assess the local retention of formulations injected at the sciatic nerve. As demonstrated by the TEM micrographs in FIG. 32, covalent conjugation of Alexa647 did not change the morphology of supramolecular nanofibers.

For Alexa647-tagged P1, MuQP1 or MuDP1 coupling, Alexa647-NHS ester (0.2 micromoles) was added into a solution comprising the relevant peptide moiety in PBS (100 microliters, 0.5 mM). The mixture was incubated in a 1.0 mL Eppendorf tube at 25° C. for 3 h with gentle shaking (the pH control at 7.4 for the whole process). The conjugated peptide moiety (denoted by the suffix ‘Alexa-’, e.g., Alexa-P1) was purified by NAP-5 column (GE Healthcare, UK) preequilibrated with PBS buffer. Under isoflurane-oxygen anesthesia, rats were shaved and injected with 0.2 mL of test formulation. The in vivo fluorescence images were captured, and the fluorescence intensity was evaluated at predetermined time points post-injection (under brief isoflurane-oxygen anesthesia) using a Spectrum in vivo imaging system (IVIS) (PerkinElmer, MA, USA). Whole body animal images were recorded non-invasively. The 675 nm excitation filter and the 700 nm emission filter were used for the imaging. Quantitative analysis was carried out using the Live Imaging® software of the IVIS. The half-life of tissue retention was the time required for the fluorescence intensity to decrease by 50% after injection. The time point when the fluorescence intensity dropped to 50% of its maximal value corresponded to the half-life of tissue retention.

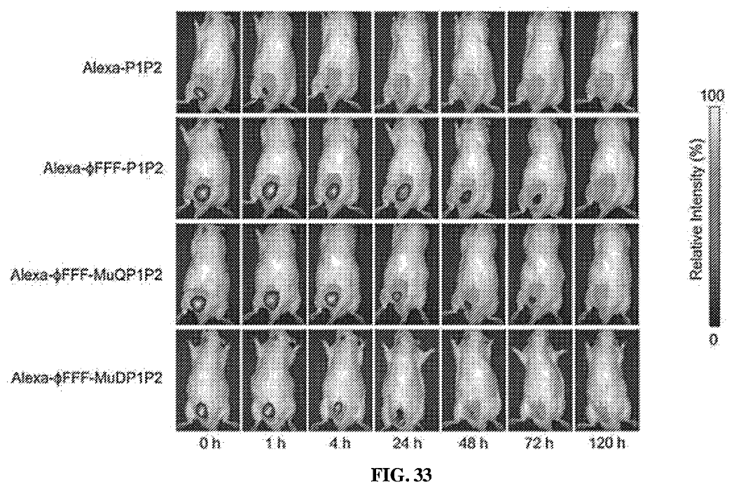

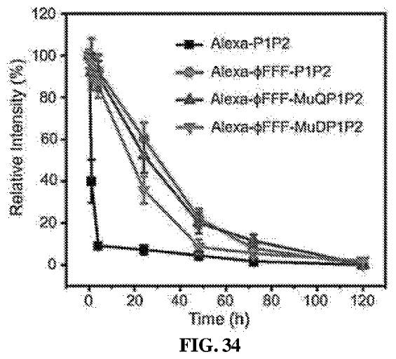

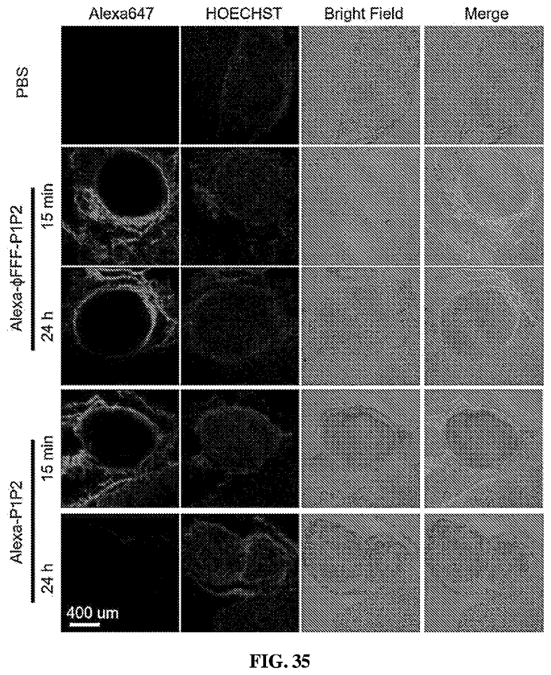

The local fluorescence intensity was monitored at predetermined intervals after injection, using an IVIS. In animals injected with Alexa-P1P2 (626.4 micromolar; peptides without hydrophobic modification), fluorescence at the site of injection dropped rapidly in the first hour, and was almost gone by 4 hours (FIGS. 33-34). This is demonstrated by the IVIS images presented in FIG. 33, and by the calculated decline in relative intensity over time, presented in FIG. 34. The tissue retention of formulations where peptides spontaneously formed supramolecular nanofibers (ϕFFF-P1P2, ϕFFF-MuQP1P2, and ϕFFF-MuDP1P2) was significantly prolonged, i.e., approximately 50% of fluorescence intensity remained after 24 hours. Laser scanning confocal microscopy of frozen sections of the sciatic nerve and surrounding tissues 24 h after injection confirmed that Alexa- ϕFFF-P1P2 was retained longer at the sciatic nerve than was Alexa-P1P2. FIG. 35 presents these confocal microscopy of frozen sections of the sciatic nerve and surrounding tissues 15 minutes and 24 hours after injection.

Laser scanning confocal microscopy was performed by the following protocol. Under brief isoflurane-oxygen anesthesia, rats were injected with 0.2 mL of test formulation (Alexa-P1P2 or Alexa- ϕFFF-P1P2), then euthanized at predetermined intervals (15 min and 24 h). Sciatic nerves together with surrounding tissues were harvested and embedded into OCT compound (VWR, PA, USA), then frozen and stored at −20° C. Sections (10 micrometers) were prepared using a cryostat microtome (Leica CM3050 S, Wetzlar, Germany) and mounted onto glass slides. Afterwards, slides were fixed with 4% paraformaldehyde for 20 min at room temperature, washed in PBS (pH 7.4) 3 times. Nuclei were stained with Hoechst33342. The slices were imaged using a Zeiss LSM 710 multi-photon confocal microscopy (Carl Zeiss AG, Oberkochen, Germany).

### Example 10

In this example, the viscosity of solutions comprising exemplary peptide moieties was determined by rheometry. The rheological properties of P1P2, ϕFFF-P1P2, ϕFFF-MuQP1P2 and ϕFFF-MuDP1P2 (626.4 micromolar) were monitored using an AR2000 rheometer (TA instruments, DE, USA) equipped with a temperature controller. A parallel plate with 20 mm diameter was used for all tests. The gap distance between the plates was 0.3 mm. Frequency sweeps ranging from 0.01 to 100 rad/s were conducted at room temperature. A constant 0.1 Pa stress was used. The complex viscosity of each sample was recorded at a frequency of 0.01 rad/s.

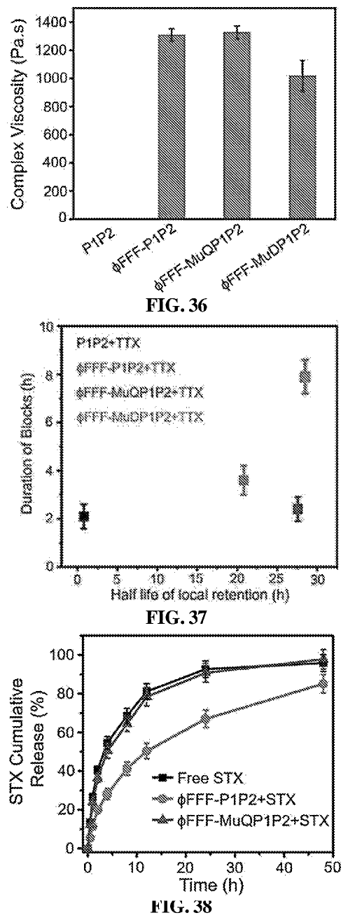

FIG. 36 presents the complex viscosity observed for each exemplary peptide moiety. ϕFFF-P1P2, ϕFFF-MuQP1P2, and ϕFFF-MuDP1P2 had much higher viscosity than P1P2 solution. The in vivo imaging described in Example 9 showed that ϕFFF-MuQP1P2, ϕFFF-MuDP1P2, Alexa- ϕFFF-MuQP1P2, and Alexa- ϕFFF-MuDP1P2 had prolonged tissue retention (FIGS. 33-34). However, their nerve block durations were much shorter than that of ϕFFF-P1P2+TTX. (ϕFFF-MuQP1P2+TTX: 2.4±0.5 h; ϕFFF-MuDP1P2+TTX: 3.6±0.6 h, p<0.01 vs. ϕFFF-P1P2+TTX; Table 5). These results suggested that although complex viscosity and local retention at the injection site were both increased in nanofibrous formulations, neither of those properties was primarily responsible for the prolongation of nerve block. Mutant peptides injected with TTX had prolonged tissue retention but relatively brief durations of block, while the duration of block from ϕFFF-P1P2+TTX was much longer, even though the tissue retention was comparable to those of the mutant peptides (FIG. 37).

### Example 11

To assess whether this approach was broadly applicable to S1SCBs, the ability of the peptide moieties of previous examples to block the release of two other agents, saxitoxin STX, pictured in FIG. 4C and dicarbamoyl saxitoxin (dcSTX) was measured. These agents block the same site (site 1) on the sodium channel, but are structurally quite dissimilar from TTX, aside from the guanidium group. STX and dc-STX are approximately twice and one-half the potency of TTX in rat sciatic nerve blockade respectively. FIG. 38 indicates the release of STX from free STX solution, ϕFFF-P1P2, and ϕFFF-MuQP1P2, in some embodiments. ϕFFF-P1P2 (ϕFFF-P1P2+STX) controlled the release of STX compared with free STX and ϕFFF-MuQP1P2+STX (FIG. 38). In the STX release test, the STX concentration in each solution was 50.1 micromolar, and other procedures were the same as for TTX.

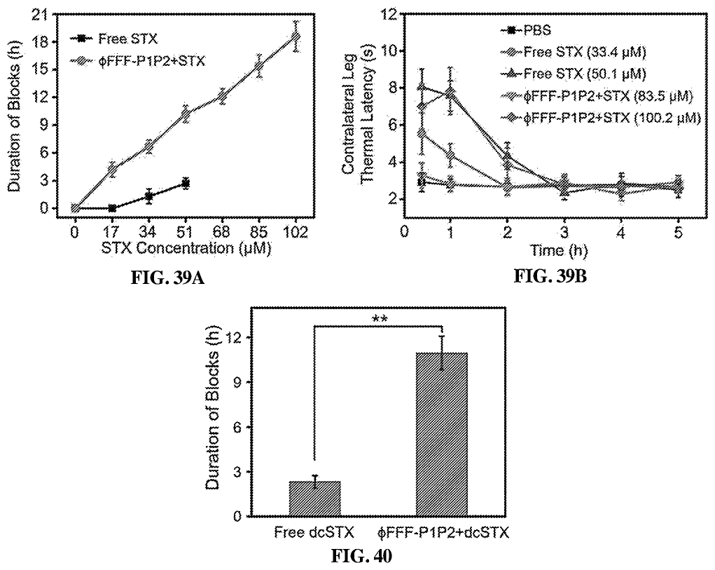

FIG. 39A presents the duration of blocks observed for various doses of STX, administered and measured as described for TTX in Example 8, above. The duration of nerve block from STX was prolonged by ϕFFF-P1P2. For example, the duration of sensory block from 50.1 μM STX was prolonged 3.8-fold to 10.2±0.9 h (n=4; p<0.01). The maximum dose of STX that could be given was increased (100.2 μM STX in ϕFFF-P1P2+STX vs. 50.1 μM of free STX), and therefore the maximum duration of sensory block was increased almost 7-fold to 18.6±1.6 h. As with TTX, the durations of sensory and motor block were similar, with motor block being ≤15% longer, especially at higher concentrations of STX. Table 7 presents these results numerically, quantifying the efficacy of STX with and without peptide moieties.

TABLE 7
Efficacy of STX formulations in sciatic nerve block.

| | | | Sensory | Motor | |
|---|---|---|---|---|---|
| | TTX | Mortality | Block Time | Block Time | P |
| Groups | (μM) | (%) | (h) | (h) | Value^{a} |
| Free STX | 16.7 | 0 | 0 | 0 | — |
| | 33.4 | 0 | 1.3 ± 1.5 | 1.5 ± 1.7 | >0.05 |
| | 50.1 | 0 | 2.7 ± 0.6 | 3.1 ± 0.6 | >0.05 |
| | 66.8 | 100 | — | — | — |
| ϕFFF-P1P2 + STX | 16.7 | 0 | 4.2 ± 0.6 | 5.1 ± 0.7 | >0.05 |
| | 33.4 | 0 | 6.7 ± 0.7 | 7.8 ± 0.6 | <0.05 |
| | 50.1 | 0 | 10.2 ± 0.9 | 11.6 ± 0.7 | <0.05 |
| | 66.8 | 0 | 12.1 ± 0.8 | 13.9 ± 0.9 | <0.05 |
| | 83.5 | 0 | 15.4 ± 1.0 | 17.6 ± 0.9 | <0.05 |
| | 100.2 | 0 | 18.6 ± 1.6 | 21.3 ± 0.9 | <0.05 |
| | 116.9 | 50 | — | — | — |
| ϕFFF-MuQP1P2 + STX | 16.7 | 0 | 0 | 0 | — |
| | 33.4 | 0 | 1.4 ± 1.7 | 1.6 ± 1.9 | >0.05 |
| | 50.1 | 0 | 3.2 ± 0.6 | 3.4 ± 0.6 | >0.05 |
| | 66.8 | 75 | — | — | — |

^{a}For the comparison of the durations of sensory and motor block. Data are means ± SD; n = 4.

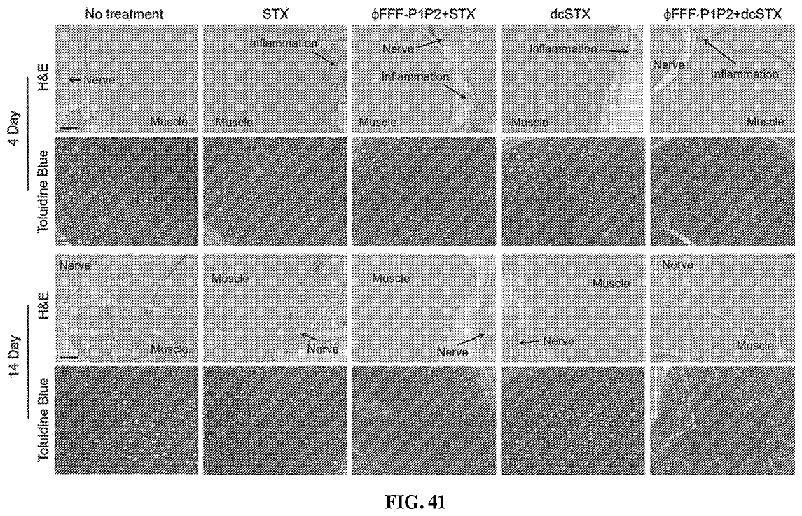

FIG. 39B presents the contralateral leg thermal latency observed for various doses of STX. The systemic toxicity of STX was reduced when delivered in ϕFFF-P1P2, as evidenced by the decrease in contralateral block and mortality (Table 7). As with TTX, ϕFFF-MuP1P2 did not prolong nerve block over that from free STX (Table 7). ϕFFF-P1P2 greatly prolonged the duration of nerve block from dc-STX (ϕFFF-P1P2+dcSTX). For example, as shown in FIG. 40, sensory nerve block from 156.1 μM dcSTX in ϕFFF-P1P2 lasted 11.0±1.1 h compared to 2.3±0.4 h for 8 μg of free dcSTX (4.8-fold prolongation). As in Example 8, four days and 14 days after injection of each formulation, the sciatic nerves and adjacent tissues were harvested, sectioned, stained with hematoxylin-eosin (H&E), and tissue reaction (inflammation and myotoxicity) was assessed. The results, showin in FIG. 41, indicate that no myotoxicity nor neurotoxicity was observed with STX or dcSTX delivered in ϕFFF-P1P2.

### Example 12

In this comparative example, MPs did not enhance the effect of bupivacaine, an amino-amide local anesthetic that binds to the same sodium channel as does TTX, but at a different site (i.e., not at site 1), located on the inner surface of the cell membrane. The bupivacaine hydrochloride (Bup) release samples were collected by the same methods described above for TTX and STX. The concentration of bupivacaine was determined by HPLC (Agilent 1260 Infinity, Agilent Technologies, Inc. CA, USA) using a C18 column (Poroshell 120 EC-C18, 4.6×100 mm, i.d. 2.7 micrometers, Agilent Technologies, Inc. CA, USA) with an acetonitrile/water (70:30) mobile phase and a flow rate of 0.5 mL/min. Bupivacaine was detected by UV absorbance at λ=254 nm.

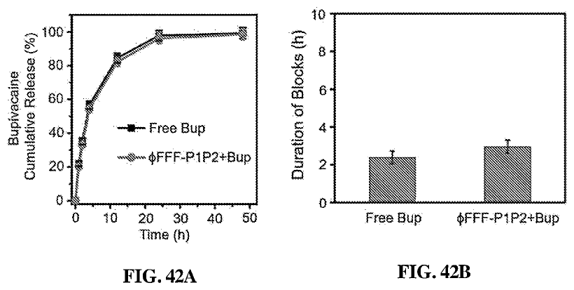

ϕFFF-P1P2 (62.6 micromolar) did not affect the release kinetics of bupivacaine hydrochloride FIG. 42A) (ϕFFF-P1: ϕFFF-P2:bupivacaine=1:1:1). The results of release experiments are reported in FIG. 42A. FIG. 42B meanwhile, demonstrates that the combination of ϕFFF-P1P2 (626.4 micromolar) with bupivacaine hydrochloride did not significantly prolong the duration of nerve block compared with free bupivacaine hydrochloride at the same concentration (15.4 mM) (no significant difference: 3.0±0.3 h from ϕFFF-P1P2+Bup vs. 2.4±0.3 h from Free Bup). Here, bupivacaine could not be used at the same concentration as the peptides, as it was too low to result in block. Consequently, a large excess of bupivacaine over peptide was used.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage. As used herein, “at %” is an abbreviation of atomic percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition, comprising:
a peptide moiety comprising a channel or receptor peptide sequence and a hydrophobic domain connected to the peptide sequence.

2. The composition of claim 1, wherein the peptide moiety comprises a channel peptide sequence.

3. The composition of claim 2, wherein the channel peptide sequence comprises a structure selected from the group consisting of: TQDYWEN (SEQ ID NO: 1), CGEWIET (SEQ ID NO: 2), TFKGWTI (SEQ ID NO: 25), and TSAGWDG (SEQ ID NO: 26).

4. The composition of any one of claims 1-3, wherein the composition further comprises an agent and a second peptide moiety comprising a second channel or receptor peptide sequence and a hydrophobic domain connected to the second peptide sequence.

5. A method, comprising delivering the composition of any one of claims 1-4 to a subject.

6. A composition, comprising:
a peptide moiety comprising a peptide sequence having a structure TQDYWEN (SEQ ID NO: 1) and a hydrophobic domain connected to the peptide sequence.

7. A composition, comprising:
a peptide moiety comprising a peptide sequence having a structure CGEWIET (SEQ ID NO: 2) and a hydrophobic domain connected to the peptide sequence.

8. The composition of claim 7, wherein the hydrophobic domain is connected to a terminus of the peptide sequence.

9. The composition of any one of claims 7 or 8, wherein the hydrophobic domain comprises a hydrophobic peptide sequence, comprising between 3 and 10 hydrophobic amino acid residues selected from: alanine, isoleucine, leucine, methionine, phenylalanine, tryptophan, tyrosine, valine, glycine, and proline.

10. The composition of any one of claims 7-9, wherein the hydrophobic domain comprises a species of type (I): 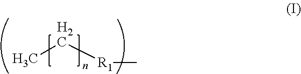
where R₁ is a carbonyl carbon or R₁ is an amide nitrogen.

11. The composition of any one of claims 7-10, wherein the hydrophobic domain comprises a species of type (II): 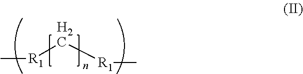
where R¹ and R² are each, independently selected from a carbonyl carbon and an amide nitrogen, and wherein R₂ is bonded to a second peptide sequence comprising a sequence having the structure of TQDYWEN (SEQ ID NO: 1) or CGEWIET (SEQ ID NO: 2).

12. The composition of any one of claims 7-11, wherein the hydrophobic domain is an N-terminal modification of the peptide sequence.

13. The composition of any one of claims 7-11, wherein the hydrophobic domain is a C-terminal modification of the peptide sequence.

14. The composition of any one of claims 7-13, wherein the hydrophobic peptide sequence is connected to the peptide sequence at a first terminus of the hydrophobic peptide sequence.

15. The composition of any one of claims 7-14, wherein the hydrophobic peptide sequence is connected to a second peptide sequence comprising a sequence having the structure of TQDYWEN (SEQ ID NO: 1) or CGEWIET (SEQ ID NO: 2).

16. The composition of claim 15, wherein the hydrophobic peptide sequence is connected to the second peptide sequence at a second terminus of the hydrophobic peptide sequence.

17. The composition of any one of claims 7-16, wherein the peptide moiety is assembled into a supramolecular nanostructure.

18. The composition of claim 17, wherein the supramolecular nanostructure comprises a first species of peptide moiety comprising a sequence having the structure of TQDYWEN (SEQ ID NO: 1), and comprises a second species of peptide moiety comprising a sequence having the structure of CGEWIET (SEQ ID NO: 2).

19. The composition of any one of claims 7-18, wherein the supramolecular nanostructure is a supramolecular nanofiber.

20. The composition of any one of claims 7-19, wherein the peptide moiety is associated with an agent.

21. The composition of claim 20, wherein the agent comprises an anesthetic.

22. The composition of any one of claims 20 or 21, wherein the agent comprises a sodium channel blocker.

23. The composition of claim 22, wherein the agent is the sodium channel blocker.

24. The composition of any one of claims 20-23, wherein the agent comprises at least one of tetrodotoxin and saxitoxin.

25. The composition of any one of claims 20-24, wherein the supramolecular nanostructure is configured to retain at least 50% of the agent for more than 3 hours when dialyzed at 37° C.

26. The composition of any one of claims 20-25, wherein the agent is released in vivo.

27. The composition of any one of claims 7-26, wherein circular dichroism analysis of the supramolecular nanostructure at a 20 micromolar concentration produces a mean residue ellipticity of less than or equal to −10 and greater than or equal to 10 for wavelengths between 190 nm and 260 nm.

28. A composition, comprising:
a supramolecular structure, comprising:
a first peptide moiety comprising a first sodium channel peptide sequence and a first hydrophobic domain; and
a second peptide moiety comprising a second sodium channel peptide sequence and a second hydrophobic domain,
wherein the first hydrophobic domain is associated with the second hydrophobic domain via a hydrophobic interaction.

29. The composition of claim 28, further comprising an agent associated with the first peptide sequence and the second peptide sequence.

30. The composition of claim 29, wherein the agent comprises an anesthetic.

31. The composition of any one of claims 28-30, wherein the agent comprises a sodium channel blocker.

32. The composition of any one of claims 28-31, wherein the agent comprises at least one of tetrodotoxin and saxitoxin.

33. The composition of any one of claims 28-32, wherein the supramolecular nanostructure is configured to retain at least 50% of the agent for more than 3 hours when dialized at 37° C.

34. The composition of any one of claims 28-33, wherein the supramolecular nanostructure is a supramolecular nanofiber.

35. The composition of any one of claims 28-34, wherein circular dichroism analysis of the supramolecular nanostructure at a 20 micromolar concentration produces a mean residue ellipticity of less than or equal to −10 and greater than or equal to 10 for wavelengths between 190 nm and 260 nm.

36. The composition of any one of claims 28-35, wherein the supramolecular nanostructure comprises bolaamphiphiles.

37. The composition of any one of claims 28-36, wherein first peptide moiety and the second peptide moiety are the same.

38. A method, comprising:
associating an agent with a supramolecular nanofiber comprising a first peptide moiety and a second peptide moiety; and
releasing the agent from the supramolecular nanofiber over a period of at least 3 hours.

39. A method, comprising:
associating an agent with a supramolecular nanostructure comprising a first peptide moiety comprising a first peptide sequence and a first hydrophobic domain, and a second peptide moiety comprising a second peptide sequence and a second hydrophobic domain, wherein the first hydrophobic domain is associated with the second hydrophobic domain via a hydrophobic interaction; and
releasing the agent from the supramolecular structure over a period of at least 3 hours.

40. The method of claim 39, wherein the agent comprises an anesthetic.

41. The method of any one of claims 39 or 40, wherein the agent comprises a sodium channel blocker.

42. The method of any one of claims 39-41, wherein the agent comprises at least one of tetrodotoxin and saxitoxin.

43. The method of any one of claims 39-42, wherein the supramolecular nanostructure is configured to retain at least 50% of the agent for more than 3 hours when dialyzed at 37° C.

44. The method of any one of claims 39-43, further comprising releasing the agent in vivo.

45. The method of any one of claims 39-44, wherein circular dichroism analysis of the supramolecular nanostructure at a 20 micromolar concentration produces a mean residue ellipticity of less than or equal to −10 and greater than or equal to 10 for wavelengths between 190 nm and 260 nm.

46. The method of any one of claims 39-45, wherein the supramolecular nanostructure comprises bolaamphiphiles.


SEQUENCE LISTING (26 sequences)

SEQ ID NO: 1 - protein, 7 aa, synthetic construct
TQDYWEN

SEQ ID NO: 2 - protein, 7 aa, synthetic construct
CGEWIET

SEQ ID NO: 3 - protein, 5 aa, synthetic construct
FFFLL

SEQ ID NO: 4 - protein, 7 aa, synthetic construct
CGQWIET

SEQ ID NO: 5 - protein, 13 aa, synthetic construct
FFFLLGTQDYWQN

SEQ ID NO: 6 - protein, 13 aa, synthetic construct
FFFLLGCGQWIET

SEQ ID NO: 7 - protein, 7 aa, synthetic construct
TQDYWDN

SEQ ID NO: 8 - protein, 7 aa, synthetic construct
CGDWIET

SEQ ID NO: 9 - protein, 13 aa, synthetic construct
FFFLLGTQDYWDN

SEQ ID NO: 10 - protein, 13 aa, synthetic construct
FFFLLGCGDWIET

SEQ ID NO: 11 - protein, 13 aa, synthetic construct
FFFLLGQDNWTYE

SEQ ID NO: 12 - protein, 5 aa, synthetic construct
YFYLL

SEQ ID NO: 13 - protein, 7 aa, synthetic construct
QDNWTYE

SEQ ID NO: 14 - protein, 7 aa, synthetic construct
EGWEICT

SEQ ID NO: 15 - protein, 8 aa, synthetic construct
GTQDYWEN

SEQ ID NO: 16 - protein, 8 aa, synthetic construct
GCGEWIET

SEQ ID NO: 17 - protein, 13 aa, synthetic construct
FFFLLGTQDYWEN

SEQ ID NO: 18 - protein, 13 aa, synthetic construct
FFFLLGCGEWIET

SEQ ID NO: 19 - protein, 13 aa, synthetic construct
YFYLLGTQDYWEN

SEQ ID NO: 20 - protein, 13 aa, synthetic construct
YFYLLGCGEWIET

SEQ ID NO: 21 - protein, 13 aa, synthetic construct
FFFLLGEGWEICT

SEQ ID NO: 22 - protein, 6 aa, synthetic construct
FFFLLG

SEQ ID NO: 23 - protein, 6 aa, synthetic construct
YFYLLG

SEQ ID NO: 24 - skipped

SEQ ID NO: 25 - protein, 7 aa, synthetic construct
TFKGWTI

SEQ ID NO: 26 - protein, 7 aa, synthetic construct
TSAGWDG